(19) United States
(12) Patent Application Publication
(10) Pub. No.: US 2022/0175369 A1
(43) Pub. Date: Jun. 9, 2022

(54) Surgical Fastening Device

(71) Applicant: Surgimatix, Inc., Eld Grove Village, IL (US)

(72) Inventors: Jafar S. Hasan, Oak Brook, IL (US); Adam A. Saban, Sioux Falls, SD (US); Gary M. Kobylewski, Hoffman Estates, IL (US)

(73) Assignee: Surgimatix, Inc., Eld Grove Village, IL (US)

(21) Appl. No.: 17/601,370

(22) PCT Filed: Apr. 3, 2020
(86) PCT No.: PCT/US2020/026660

§ 371 Date: Oct. 4, 2021

Related U.S. Application Data
(60) Provisional application No. 62/829,167, filed on Apr. 4, 2019.

(51) Int. Cl. A61B 17/064; A61B 17/068; A61B 17/00

(52) U.S. Cl. CPC A61B 17/064; A61B 17/068; A61B 2017/0649; A61B 2017/0645; A61B 17/00234

(57) ABSTRACT

In accordance with one aspect of the disclosure, a surgical fastener is disclosed. The surgical fastener includes a base at a proximal end of the surgical fastener. A first arm and a second arm extend helically from the base about a longitudinal axis towards a distal end of the surgical fastener. A first needle capture zone is disposed on a distal portion of the first arm, and a second needle capture zone is disposed on a distal portion of the second arm. The first needle capture zone and the second needle capture zone are configured to engage with a respective first needle and a second needle when the first and the second needle are extending from a fastener deployment device and disengage with the respective first and second needle when the first and the second needle are retracting into the fastener deployment device.

### CROSS-REFERENCE TO RELATED APPLICATION

This application is an International Patent Application under the Patent Cooperation Treaty claiming priority to U.S. Provisional Application Ser. No. 62/829,167 filed on Apr. 4, 2019.

### TECHNICAL FIELD

The present disclosure generally relates to medical devices, and more particularly relates to medical fastening devices for fastening tissue or prosthetic material.

### BACKGROUND ART

The fastening of tissues has long been a need in the medical industry, and correspondingly, a finite number of fastening devices have been developed for different applications and uses. Among these devices are laparoscopic fastening devices, or tackers, which are often used with minimally invasive procedures such as laparoscopic repair of hernias, and the like. A typical laparoscopic procedure involves the insertion of thin, elongated instruments into relatively small incisions or access ports in the abdomen to access hernia defects in the abdominal wall from the inside. Moreover, the laparoscopic instruments are used to position a prosthetic mesh over the defect and fasten the prosthetic mesh against the inner abdominal wall using tacks, fasten tissues together, or the like. The small incisions provide for a minimally invasive surgery leading to less discomfort, and faster recovery. One such surgical procedure may include repair of a hernia that involves making small incisions and applying fasteners within an abdominal cavity.

Some laparoscopic tackers provide a relatively thin and elongated tubular member containing deployable tacks and having either an end-firing mechanism or a side-firing mechanism positioned at the distal tip thereof. Some drawbacks of conventional laparoscopic tackers include rigid and sharp tacks migrating from the location of installation, and difficulty in contorting the installation site (e.g., an abdominal wall) to achieve a proper insertion angle

For example, in the case of metal, coil-like tacks, these tacks may cause irritation or pain to the patient, become dislodged from the abdominal wall, or cause other complications post surgery. To address such drawbacks associated with metal tacks, absorbable tacks have been developed and employed. Absorbable tacks are designed to be eventually absorbed by the body, and thus, cause less irritation or pain to the patient over time. However, absorbable tacks also tend to provide holding or tensile strength that is less than optimal. In such cases, suturing the hernia defects or suturing prosthetic mesh to the abdominal wall may prove to be more effective. However, the relatively complex nature involved with suturing makes it difficult to use sutures on hernia defects via laparoscopic or otherwise minimally invasive procedures.

Accordingly, there is a need for minimally invasive or laparoscopic means of tissue fastening or installing sutures in tissue which substantially facilitates the installation process for the surgeon or user. There is also a need for a medical fastening device which provides a more effective and reliable means for closing tissue and/or fastening prosthetic mesh to tissue. Furthermore, there is a need for a medical fastening device which employs fasteners that reduce irritation, pain, and other complications to the patient without adversely affecting tissue holding strength.

### DISCLOSURE OF INVENTION

In accordance with one aspect of the disclosure, a surgical fastener is disclosed. The surgical fastener includes a base at a proximal end of the surgical fastener. A first arm and a second arm extend helically from the base about a longitudinal axis towards a distal end of the surgical fastener. A first needle capture zone is disposed on a distal portion of the first arm, and a second needle capture zone is disposed on a distal portion of the second arm. The first needle capture zone and the second needle capture zone are configured to engage with a respective first needle and a second needle when the first needle and the second needle are extending from a fastener deployment device and disengage with the respective first and second needle when the first and the second needle are retracting into the fastener deployment device.

In accordance with another aspect of the disclosure, a method of installing a surgical fastener in a tissue is disclosed. The method includes loading the surgical fastener at a needle interface of a fastener deployment device, extending a first needle and a second needle along a first helical arm and a second helical arm of the surgical fastener, engaging the first needle with a first needle capture zone disposed on a distal portion of the first helical arm, engaging the second needle with a second needle capture zone disposed on a distal portion of the second helical arm, pulling, by the first and second needle capture zones, the surgical fastener through the tissue, and retracting the first and second needles to disengage the first and second needles from the respective first and second needle capture zones.

Another embodiment takes the form of a fastener deployment device. The fastener deployment device includes a first spiral needle and a second spiral needle, with the first and second spiral needles disposed at a distal end of the fastener deployment device. The fastener deployment device further includes a drive shaft extending along a longitudinal axis, with the drive shaft being disposed within an outer sleeve and coupled to the first and second spiral needles, and a drive mechanism at a proximal end of the fastener deployment device, with the drive mechanism being operatively coupled to the drive shaft and configured to extend both the first and second spiral needles axially along and radially around the longitudinal axis simultaneously.

In accordance with another aspect of the disclosure, a drive mechanism for a fastener deployment device is provided for. The drive mechanism includes a lead screw having a distal end and a proximal end, wherein the lead screw extends along a longitudinal axis, is operatively coupled to a drive shaft at the distal end, and includes a threaded portion on an outer circumference of the lead screw. The drive mechanism further includes a guide nut fixedly mounted within a housing, with the guide nut being configured to receive the threaded portion of the lead screw, and an actuation device configured to apply an actuation force along the longitudinal axis to the proximal end of the lead screw. The actuation force causes the threaded portion of the lead screw to simultaneously axially advance along and rotate around the longitudinal axis within the guide nut.

Yet another embodiment takes the form of an articulation device for a fastener deployment device. The articulation device includes a flexible drive shaft configured to receive an insertion input from an actuator at a proximal end and transmit the insertion input to a needle interface to install a surgical fastener at a distal end. The articulation device further includes a proximal outer sleeve, an articulation tendon connecting the distal end to the proximal outer sleeve, and an inner articulation tube having a plurality of articulation joints along a length of the inner articulation tube, the inner articulation tube being disposed around the flexible drive shaft and being disposed at least partially within the proximal outer sleeve. The articulation device further includes an articulation actuator configured to retract the proximal outer sleeve away from the distal end to sequentially expose the articulation joints in the plurality of articulation joints to cause the distal end, pulled by the articulation tendon, to bend at the exposed articulation joints.

These and other aspects and features of the disclosure will be better understood upon reading the following detailed description when taken into conjunction with the accompanying drawings.

### BRIEF DESCRIPTION OF DRAWINGS

FIG. 1 is a perspective view of a first surgical fastener, in accordance with the teachings of the present disclosure;

FIG. 2 is a perspective view of a second surgical fastener, in accordance with the teachings of the present disclosure;

FIG. 3 is a perspective view of a third surgical fastener, in accordance with the teachings of the present disclosure;

FIG. 4 is a perspective view of a fourth surgical fastener, in accordance with the teachings of the present disclosure;

FIG. 5 is a perspective view of a fifth surgical fastener, in accordance with the teachings of the present disclosure;

FIG. 6 is a perspective view of a fastener deployment device, in accordance with the teachings of the present disclosure;

FIG. 7 is a perspective view of a surgical fastener adjacent to a needle interface, in accordance with the teachings of the present disclosure;

FIG. 8 is a perspective view of a surgical fastener installed on a needle interface, in accordance with the teachings of the present disclosure;

FIG. 9 depicts a method, in accordance with the teachings of the present disclosure;

FIG. 10 depicts a plurality of perspective views of installation of a surgical fastener, in accordance with the teachings of the present disclosure;

FIG. 11 depicts a perspective view and a side-view of an installed surgical fastener, in accordance with the teachings of the present disclosure;

FIG. 12 depicts an exploded view and an assembled view of components of a fastener deployment device, in accordance with the teachings of the present disclosure;

FIG. 13 depicts cross-sectional views of a drive mechanism and a needle interface in a retracted and an extended state, in accordance with the teachings of the present disclosure;

FIG. 14A depicts a perspective view of a yoke and a yoke installed with a drive mechanism, in accordance with the teachings of the present disclosure.

FIG. 14B depicts perspective views of components of a drive mechanism in a retracted and an extended state, in accordance with the teachings of the present disclosure;

FIG. 15 depicts cross-sectional views of an impulse energy drive mechanism, in accordance with the teachings of the present disclosure;

FIG. 16 depicts a cross-sectional view of an impulse energy drive mechanism during activation and close-up cross-sectional views of a trigger pawl, in accordance with the teachings of the present disclosure;

FIG. 17 depicts cross-sectional views of an impulse energy drive mechanism after installation of a surgical fastener, in accordance with the teachings of the present disclosure;

FIG. 18 depicts cross-sectional views of aspects of a continuous energy drive mechanism, in accordance with the teachings of the present disclosure;

FIG. 19 depicts unassembled components of an articulation device, in accordance with the teachings of the present disclosure;

FIG. 20 depicts an assembled articulation device in a straight state, in accordance with the teachings of the present disclosure;

FIG. 21 depicts an assembled articulation device in an articulated state, in accordance with the teachings of the present disclosure;

FIG. 22 depicts a side view of an exemplary flexible tube, in accordance with accordance with the teachings of the present disclosure;

FIG. 23 depicts a perspective view of an exemplary flexible tube, in accordance with the teachings of the present disclosure;

FIG. 24 depicts a perspective view of a two-tendon articulation device, in accordance with the teachings of the present disclosure;

FIG. 25 depicts a cross-sectional view of the two-tendon articulation device of FIG. 24, in accordance with the teachings of the present disclosure;

FIG. 26 depicts a perspective view of an articulation control portion of a fastener deployment device, in accordance with the teachings of the present disclosure;

FIG. 27 depicts a perspective view of the articulation control portion of FIG. 26 with the articulation control knob removed, in accordance with the teachings of the present disclosure;

FIG. 28 depicts an exploded view of the articulation control portion of FIG. 27, in accordance with the teachings of the present disclosure;

FIG. 29 depicts the fastener deployment device of FIG. 26 in an unarticulated position, in accordance with the teachings of the present disclosure;

FIG. 30 depicts the fastener deployment device of FIG. 26 in a partially articulated position, in accordance with the teachings of the present disclosure; and

FIG. 31 depicts the fastener deployment device of FIG. 27 in a fully articulated position, in accordance with the teachings of the present disclosure.

While the present disclosure is susceptible to various modifications and alternative constructions, certain illustrative embodiments thereof have been shown in the drawings and will be described below in detail. It should be understood, however, that there is no intention to limit the present invention to the specific forms disclosed, but on the contrary, the intention is to cover all modifications, alternative constructions and equivalents falling within the spirit and scope of the present disclosure.

### MODES FOR CARRYING OUT THE INVENTION

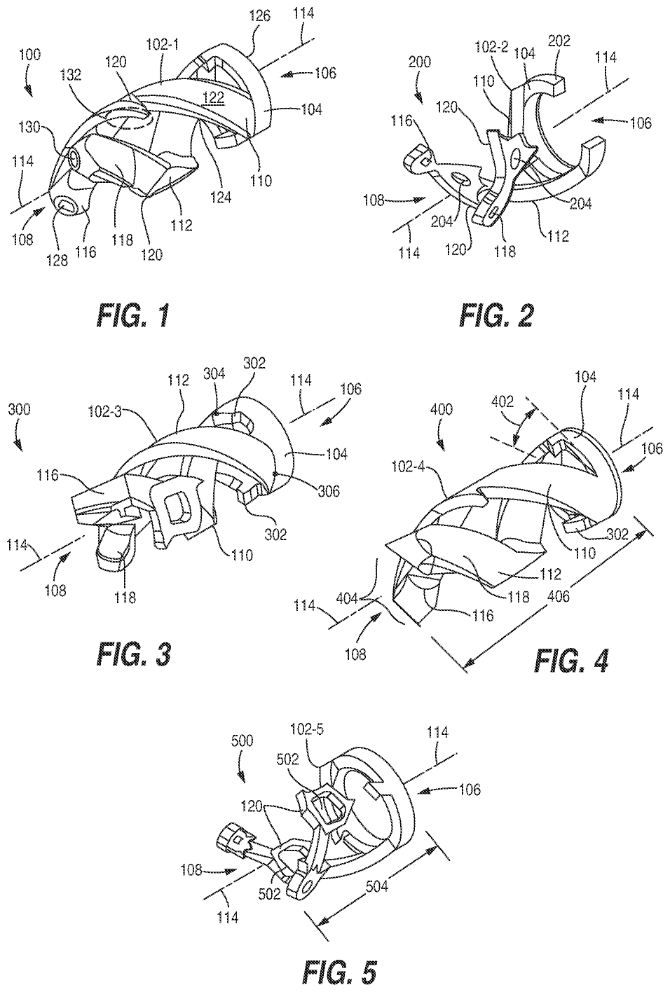

Referring now to the drawings, and with specific reference to FIG. 1, FIG. 1 depicts a perspective view of a first surgical fastener, in accordance with the teachings of the present disclosure. In particular, FIG. 1 depicts the perspective view 100 of the surgical fastener 102-1. The FIGS. 1-5 depict the surgical fasteners 102-1 through 102-5 that share many of the same features with each other and are used to teach the varying embodiments of the surgical fasteners 102 disclosed herein. As such, like-numbered components indicate similar features among the various surgical fasteners 102. Further, a convention used throughout the detailed description has a proximal direction on the right as depicted in the figures, and a distal direction on the left as depicted in the figures, unless otherwise noted. Although the term surgical fastener is used throughout to describe the various fixation devices, it is understood that the surgical fasteners 102 disclosed herein are generally flexible, similar to a suture.

The surgical fasteners 102 disclosed herein may be used to fasten a mesh to a tissue, a first piece of tissue to a second piece of tissue, or the like. In various embodiments, the surgical fastener 102 may be realized as a resorbable fastener. The surgical fastener 102 may also be realized as a flexible fastener, for example by producing it from polymeric materials. A resorbable fastener may be manufactured from components and materials configured to dissolve when installed in a tissue. Further, a flexible fastener may aide in patient comfort, permit moderate flexing of the surgical fastener 102 when installed into the tissue without tearing, and the like. In some embodiments, the surgical fasteners 102 are manufactured from a permanent or non-resorbable material, such as polypropylene, polyester, or nylon as possible non-limiting examples.

Returning to the discussion of FIG. 1, the surgical fastener 102-1 includes a base 104 at a proximal end 106 of the surgical fastener 102-1. As discussed more fully below, the base 104 may be a circular base that is realized as a full circle (e.g., a circular ring 126), a semi-circle 202 of FIG. 2, or the like. In yet other embodiments, the base 104 may be realized by any complimentary shape associated with a drive shaft. In some embodiments, the surgical fasteners 102 are installed in a laparoscopic surgery with a fastener deployment device that includes a drive shaft configured to reach a fastening site within a patient while being controlled (e.g., actuated) from a drive mechanism outside the patient. With a desire to make small incisions in the patient, the size and the shape of the drive shaft may be changed. Accordingly, the size and the shape of a surgical fastener 102 may correspondingly change (e.g., to an oval, a rectangle, a polygon) to be complementary in shape and size to the drive shaft. Presently, the surgical fasteners 102 are envisioned to be compatible in shape with a drive shaft with round cross-sectional profile.

The surgical fastener 102-1 further includes a first arm 110 and a second arm 112. The first arm 110 and the second arm 112 extend helically from the base 104 about a longitudinal axis 114 towards a distal end 108 of the surgical fastener 102-1. The first arm 110 and the second arm 112 include an outer circumferential surface 122 and also includes side surfaces 124. Both the first arm 110 and the second arm 112 extend helically about the longitudinal axis 114 in the same direction, such that they form a double helix shape. In some embodiments, the first arm 110 and the second arm 112 form a circular double helix, with the outer circumferential surface 122 having a constant radius measurement from the longitudinal axis 114 for at least a portion of the length of the surgical fastener 102.

While the surgical fasteners 102-1 through 102-5 are depicted and described as having a first arm 110 and a second arm 112, in some embodiments, the surgical fasteners 102 include more than two arms. For example, a surgical fastener 102 having three (3) arms may include three arms extending helically from the base 104 from three points that are spaced apart from each other. As such, a needle interface used to install the surgical fastener 102 may be adapted to include the like number of needles as there are arms on the surgical fastener 102.

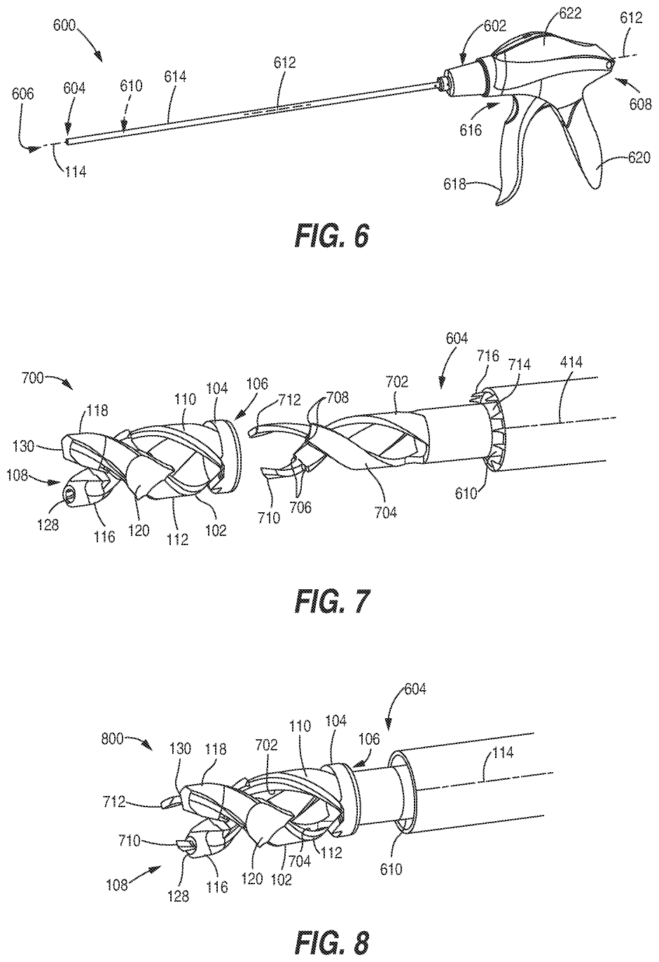

The surgical fastener 102 further includes a first needle capture zone 116 disposed on a distal portion of the first arm 110 and a second needle capture zone 118 disposed on a distal portion of the second arm 112. The first needle capture zone 116 and the second needle capture zone 118 are configured to engage with a respective first needle and a second needle (e.g., spiral needles 702, 704 of FIG. 7) when the first and the second needles are extending from a fastener deployment device (e.g., fastener deployment device 602 of FIG. 6) and disengage with the respective first and second needles when the first and the second needles are retracting into the fastener deployment device.

In general, a needle capture zone (e.g., 116, 118) serves to engage with a respective needle (e.g., 702, 704) when installing the surgical fastener 102. The needle capture zones are disposed on distal portions of the arms (e.g., 110, 112) at the distal end 108 of the surgical fastener 102. The engagement between the needle capture zones and the generally rigid needles allows for the extending needles to pull, from the distal end 108 of the surgical fastener 102, the surgical fastener 102 through mesh and/or tissue.

In some embodiments, the surgical fastener 102 is configured for the tips of the first and second needles to extend through the respective first and second needle capture zones 116, 118 to penetrate a tissue. For example, the first needle capture zone 116 may include a first needle hole 128 and the second needle capture zone 118 may include a second needle hole 130. The first needle hole 128 and the second needle hole 130 may be sized such that a piercing tips of the first needle and the second needle may pass through the respective first and second needle holes 128, 130. An inside surface of the needle capture zones 116, 118 may include a complimentary shape to promote engagement with the first and second needles. In yet other embodiments disclosed in more detail below, the needle capture zones are configured to enclose the tips of the needles such that the needles do not extend through the surgical fastener 102. In such embodiments, a tip of the surgical fastener 102 pierces the tissue or mesh.

The surgical fastener 102 may further include a retention device 120. The retention device 120 prevents retraction of the surgical fastener 102 once installed. As the needles (e.g., 702, 704) retract after installation of the surgical fastener 102 into a tissue or mesh, the retention device 120 maintains the surgical fastener 102 within the tissue, and the needles disengage (e.g., separate from) the needle capture zones 116, 118. The retention device 120 may be realized as a canted protrusive profile or other similar shape that presents an increased frictional force when retracting as compared to the frictional force experienced when being pulled through the tissue.

In some embodiments, the outer circumferential surface 122 of the first arm 110 and the second arm 112 is smooth. In such an embodiment, the retention device 120 may be disposed on the side surface 124 of each of the arms 110, 112. Thus, the retention device 120 extends into an open space 132 (indicated by a dashed circle in FIG. 1) between the first arm 110 and the second arm 112. The retention device 120 may be realized on both side surfaces 124 (e.g., side surfaces opposing each other) of each arm 110, 112.

FIG. 2 is a perspective view of a second surgical fastener, in accordance with the teachings of the present disclosure. In particular, FIG. 2 depicts the perspective view 200 of the second surgical fastener 102-2. The second surgical fastener 102-2 includes many of the same features as the first surgical fastener 102-1, including the first arm 110 and the second arm 112, the first needle capture zone 116, the second needle capture zone 118, and the retention device 120. The second surgical fastener 102-2 extends along the longitudinal axis 114 from the proximal end 106 to the distal end 108. The aspects disclosed in conjunction with the discussion of the surgical fastener 102-2 may be applied to the other surgical fasteners 102 disclosed herein.

In some embodiments, the base 104 of the surgical fastener 102 is realized by the semi-circle 202. The semi-circle 202 extends through greater than 180 degrees of arc (e.g., is more than a half-circle) to provide a grip about a drive shaft. The opening of the semi-circle 202 permits the base 104 to be installed about a drive shaft from a direction transverse to the longitudinal axis 114.

Also depicted in the view 200 is a retention device 120 having a recess 204. The recess 204 may permit a reduced insertion force of the surgical fastener 102 through the tissue as the sides of the retention device 120 may temporarily deform into the space of the recess 204 then spring back to a pre-deformation shape upon completion of fastener insertion in order to prevent fastener retraction.

FIG. 3 is a perspective view of a third surgical fastener, in accordance with the teachings of the present disclosure. In particular, FIG. 3 depicts the perspective view 300 of the third surgical fastener 102-3. Like the surgical fastener 102-2, the surgical fastener 102-3 also includes many of the same features as the surgical fastener 102-1. The aspects disclosed in conjunction with the discussion of the surgical fastener 102-3 may be applied to the other surgical fasteners 102 disclosed herein.

The surgical fastener 102-3 may also include a de-rotation locking feature 302. The de-rotation locking feature 302, as depicted in the FIGS. 1 and 3-5 but only labeled in FIGS. 3 and 4 for clarity, extends from the base 104 towards the distal end 108 in an angular, or canted, fashion in order to allow the de-rotation locking feature 302 to slide along the fastening interface near the completion of fastener insertion as the base 104 rotates flush against the fastening interface (e.g., a tissue or a mesh that the surgical fastener 102 is being installed). The shape of the de-rotation locking feature would then provide resistance against the fastening interface to prevent de-rotation (e.g., backing out in a retraction direction) about the longitudinal axis 114 of an installed surgical fastener 102. It is further envisioned that a surgical suture 102 includes a single de-rotation locking feature 302 or a plurality of de-rotation locking features 302 disposed on the base 104. For example, the surgical fastener 102-2 of FIG. 2 may include a de-rotation locking feature 302 on a portion of the base 104 (e.g., the semi-circular base 202) that connects the first arm 110 and the second arm 112, or a de-rotation locking feature 302 on end-portions of the semi-circular base 202 at the opening of the semi-circular base 202.

As also seen in the view 300, the first arm 110 extends from the base 104 from a first point 304 and the second arm 112 extends from the base 104 from a second point 306. The first point 304 may be disposed across from the second point 306. Thus, with both the first arm 110 and the second arm 112 helically extending about the longitudinal axis 114 in the same direction and at the same angle, the first arm 110 and the second arm 112 are inserted through portions of the tissue that are physically separated from each other to increase the surgical fastener's ability to remain installed within the tissue. This increased distance permits the arms 110, 112 of the surgical fastener 102 to engage with and be inserted through tissue that is physically separated from each other. This increases the surface area of the tissue that the surgical fastener 102 interacts with to provide for a stronger retention within the tissue.

FIG. 4 is a perspective view of a fourth surgical fastener, in accordance with the teachings of the present disclosure. In particular, FIG. 4 depicts the perspective view 400 of the fourth surgical fastener 102-4. Like the surgical fastener 102-2 and 102-3, the surgical fastener 102-4 also includes many of the same features as the surgical fastener 102-1. The aspects disclosed in conjunction with the discussion of the surgical fastener 102-4 may be applied to the other surgical fasteners 102 disclosed herein. As depicted in the view 400, the surgical fastener 102-4 includes a pitch 402, piercing tips 404, and a length 406.

The first arm 110 extends from the base 104 at a pitch 402. The pitch 402 is depicted as an angle that the arm extends from the base, but the pitch 402 may also be realized by a length in the axial direction that the arms 110, 112 extend through before completing a complete revolution about the longitudinal axis 114. Both the first arm 110 and the second arm 112 may extend from the base 104 at the same pitch 402, extend from points disposed across from each other (e.g., points 304, 306), and as such, may present a double helix shape. Although other values of the pitch 402 may be used, in one embodiment, the arms 110, 112 may complete one full revolution about the longitudinal axis across a length that is multiple of two to four times the diameter of the base. Thus, for a surgical fastener 102 having a base 104 with a diameter of four millimeters, the surgical fastener 102 may have arms 110, 112 that have a pitch 402 that corresponds to completing one full revolution about the longitudinal axis 114 across a length 406 between the proximal end 106 and the distal end 108 ranges from eight (8) and sixteen (16) millimeters.

The pitch 402 of the arms of the surgical fastener 102 may be complementary to the pitch of the needles of the fastener deployment device. Thus, during installation of the surgical fastener 102, the needles and the arms are maintained in alignment with each other as the surgical fastener 102 penetrates the tissue.

Also depicted in the view 400 are the piercing tips 404. The piercing tips 404 are disposed on the distal ends of the first and second arms 110, 112 (e.g., at the distal ends of the first and second needle capture zones 116, 118). The first and second needle capture zones 116, 118 are configured to enclose the respective tips of the first and second needles. As compared to the embodiment discussed in conjunction with the surgical fastener 102-1, the piercing tips 404 of the surgical fastener 102-4 perform the piercing of the tissue. In embodiments using the piercing tips 404, the tips of the needles may be dull, or rounded, tips to prevent inadvertent piercing of the surgical fastener 102.

In some embodiments, surgical fastener 102 is molded from a polymer base material. The mold may produce a surgical fastener with different thickness across the length 406 of the surgical fastener 102. For example, a needle capture zone 116, 118 having piercing tips 404 may be thicker at the needle capture zones 116, 118 than the thickness of the arms 110, 112. This varying thickness allows for more rigidity in the area of the piercing tips 404 and more flexibility along the area of the arms 110, 112. Other aspects of the surgical fastener 102 may also have a thicker dimension. For example, the base 104 may be molded to have a thickness greater than the thickness of the arms 110, 112.

In other embodiments, the surgical fastener 102 is a manufactured from cylindrical tube of polymer, with the features of the arms, base, needle capture zones, and the like laser-cut into the cylindrical tube. In such embodiments manufactured from a cylindrical tube, the thickness across the surgical fastener 102 may be uniform. As such, it may be preferred to install the surgical fasteners 102 with the tips of the needles piercing the tissue or mesh, rather than having a surgical fastener 102 having piercing tips 404. Surgical fasteners 102 manufactured by laser-cutting a cylindrical tube of polymer are depicted at least in part in FIGS. 2 and 5.

The surgical fastener 102 further includes a length 406. The length 406 is measured between the proximal end 106 and the distal end 108 and may correspond to a linear distance the needles extend beyond a distal tip of the fastener deployment device during installation. Thus, a full extension of the needles corresponds with the base 104 abutting against an outer surface of the fastening interface (e.g., a tissue or a mesh) the surgical fastener 102 is installed against.

FIG. 5 is a perspective view of a fifth surgical fastener, in accordance with the teachings of the present disclosure. In particular, FIG. 5 depicts the perspective view 500 of the fifth surgical fastener 102-5. Like the surgical fasteners 102-2, 102-3, and 102-4, the surgical fastener 102-5 also includes many of the same features as the surgical fastener 102-1. The aspects disclosed in conjunction with the discussion of the surgical fastener 102-5 may be applied to the other surgical fasteners 102 disclosed herein. As depicted in the view 500, the surgical fastener 102-4 further includes an enlarged recess 502 and a decreased length 504.

As compared to the recess 204 of the retention device 120 discussed in conjunction with FIG. 2, the surgical fastener 102-5 includes the retention device 120 with an enlarged recess 502. The enlarged recess 502 permits further deformation of the retention devices 120 during installation of the surgical fastener 102-5.

The view 500 further depicts the decreased length 504. As compared to the length 406 discussed in conjunction with FIG. 4, the decreased length 504 provides for a shorter surgical fastener 102 than is presented by the surgical fastener 102-4. As such, for a given pitch of the arms 110, 112, the surgical fastener 102-5 presents about one-half of a rotation about the longitudinal axis 114 as compared to a full rotation about the longitudinal axis 114 with the surgical fastener 102-4. The surgical deployment device installing the surgical fasteners of varying lengths may be configured to alter the drive mechanism that extends the needles to match the length (406, 504) of the surgical fastener 102. In such an embodiment having a decreased length 504, the decreased length 504 may be realized a multiple of one to two times the diameter of the base 104.

FIG. 6 is a perspective view of a fastener deployment device, in accordance with the teachings of the present disclosure. In particular, the view 600 depicts the fastener deployment device 602 that may be configured to install any of the surgical fasteners 102 disclosed herein. The fastener deployment device 602 includes a distal end 606 and a proximal end 608 and generally extends along the longitudinal axis 612. A drive shaft 610 extends along the longitudinal axis 612 and is disposed within an outer sleeve 614. The longitudinal axis 612 may generally be coincident to the longitudinal axis 114, in particular when the fastener deployment device 602 is not articulated at the distal end 606 (discussed more in detail below).

At the distal end 606 is the needle interface 604, which is depicted in further detail in conjunction with FIGS. 7-8. At the needle interface 604, the fastener deployment device 602 includes a first spiral needle 702 and a second spiral needle 704. A drive mechanism 616 is disposed at the proximal end 608 of the fastener deployment device 602. The drive mechanism 616 may be any number of different styles of drive mechanisms, but in general, the drive mechanism 616 is operatively coupled to the drive shaft 610 and configured to extend both the first and second spiral needles axially along and radially around the longitudinal axis 612 simultaneously.

The fastener deployment device 602 may further include a trigger 618 that is configured to actuate the drive mechanism 616, a handle 620 which may be fixed to the housing 622. Thus in some embodiments, the trigger 618 is configured to cause the application of the actuation force. The housing 622 may contain aspects of the drive mechanism 616.

As discussed above, the fastener deployment device 602 may be configured to install any of the surgical fasteners 102 disclosed herein. For example, the fastener deployment device 602 may install a surgical fastener 102 having a base 104 at a proximal end of the surgical fastener, a first arm 110 and a second arm 112 that extend helically from the base 104, and a first and a second needle capture zone 116, 118. The first spiral needle 702 and the second spiral needle 704 are configured to engage with the respective first and second needle capture zones 116, 118 when extending from the fastener deployment device 602. The first and second spiral needles 702, 704 may further disengage from the respective first and second needle capture zones 116, 118 when the first and second spiral needles are retracting into the fastener deployment device 602.

FIG. 7 is a perspective view of a surgical fastener adjacent to a needle interface, and FIG. 8 is a perspective view of the surgical fastener installed on a needle interface, in accordance with the teachings of the present disclosure. In particular, FIG. 7 depicts the perspective view 700 of the surgical fastener 102 adjacent to and apart from the needle interface 604. The perspective view 800 depicts the surgical fastener 102 installed on the needle interface 604. As depicted in the views 700 and 800, the surgical fastener 102 includes a proximal end 106 and a distal end 108. A first arm 110 and a second arm 112 extend from the base 104. A first needle capture zone 116 and a second needle capture zone 118 are disposed on a distal portion of the respective first and second arms 110, 112. Although the needle capture zones 116, 118 are depicted as having the first and second needle holes 128, 130, it is envisioned that surgical fasteners 102 without the needle holes may be used. The surgical fastener 102 may further include a retention device 120 along the first and second arms 110, 112 between the proximal end 106 and the distal end 108.

The needle interface 604 includes the first spiral needle 702 and the second spiral needle 704. The needle interface 604 may be operatively coupled to a distal end 714 of the drive shaft 610. The first spiral needle 702 includes a first shoulder 706 and the second spiral needle 704 includes a second shoulder 708. The first and second shoulders 706, 708, may abut against an inside surface of the respective needle capture zones 116, 118. These shoulders 706, 708 are configured to transmit a force in the distal direction to assist in pulling the surgical fastener 102 through a tissue. Here, the needle interface 604 includes a first needle 702 having a first tip 710 and a second needle 704 having a second tip 712. Although depicted as sharp tips configured to penetrate a tissue, the first and second tips 710, 712 of the spiral needles 702, 704 may be realized by rounded, or dulled, tips. In such an embodiment, the distal ends (e.g., the piercing tips 404) of the surgical fastener 102 penetrate the tissue as they are pushed by the needles 702, 704 that are engaged with the needle capture zones 116, 118.

In some embodiments, the fastener deployment device 602 includes tines 716 on the distal end 714, such as depicted in the view 700. The tines 716 affix a mesh to an underlying tissue during surgical fastener 102 installations. Before the surgical fastener 102 is pulled through the mesh and/or tissue, the distal end 714 abuts against the fastening interface. The tines 716 affix an outer mesh to an underlying tissue to counteract a rotation of the mesh relative to the fastening interface imparted by the rotational aspect of the needles.

As depicted in the view 800 of FIG. 8, the surgical fastener 102 is installed on the needle interface 604. Here, the first arm 110 and the second arm 112 of the surgical fastener 102 align with and are coincident with the first spiral needle 702 and the second spiral needle 704, respectively. Further, the first needle tip 710 extends through the first needle hole 128 of the first needle capture zone 116 and the second needle tip 712 extends through the second needle hole 130 of the second needle capture zone 118. When installed, the shoulders 706, 708 of the spiral needles 702, 704 abut against an inside surface of the respective needle capture zones 116, 118.

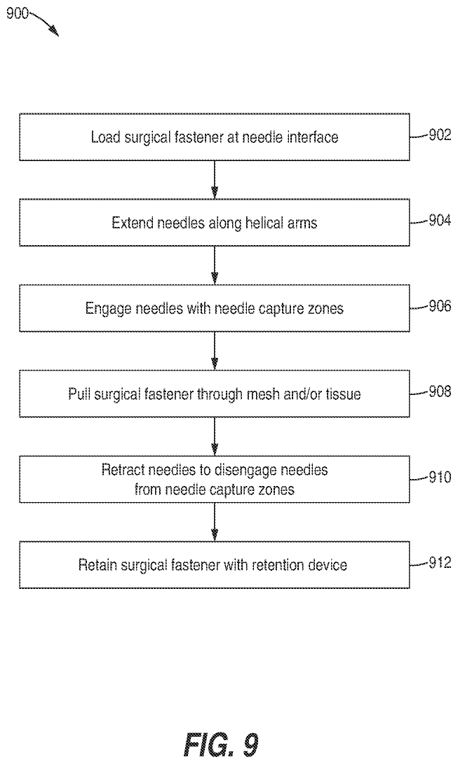

FIG. 9 depicts a method, in accordance with the teachings of the present disclosure. In particular, FIG. 9 depicts the method 900 that includes loading the surgical fastener at the needle interface at 902, extending needles along the arms at 904, engaging the needles with the needle capture zones at 906, pulling the surgical fastener through a tissue and/or mesh at 908, retracting the needles to disengage the needles from the needle capture zones at 910, and retaining the surgical fastener with the retention device at 912. The method 900 may be performed with any of the surgical fasteners 102 and the fastener deployment devices 602, 1902 disclosed herein.

In the method 900, at block 902, a surgical fastener 102 is loaded at a needle interface 604 of the fastener deployment device 602. At block 904, the first needle 702 and the second needle 704 are extended along a first helical arm 110 and a second helical arm 112, respectively, of the surgical fastener 102. At block 906, the first needle 702 is engaged with the first needle capture zone 116 that is disposed on a distal portion of the first helical arm and the second needle 704 is engaged with the second needle capture zone 118 that is disposed on the distal portion of the second helical arm. These aspects are depicted in the view 800 of FIG. 8 discussed above.

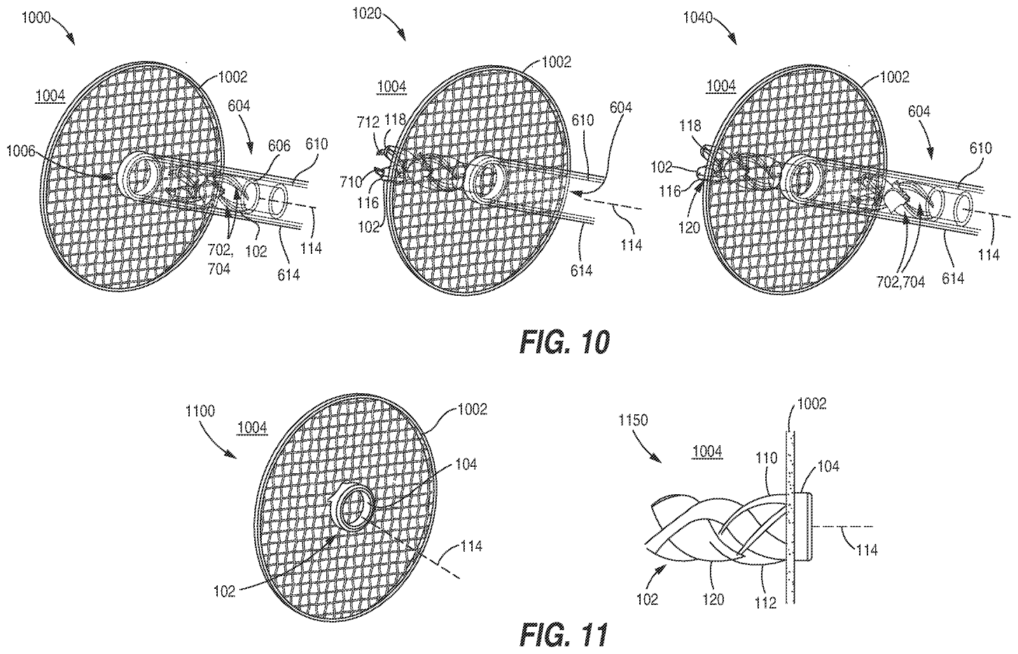

At block 908, the surgical fastener 102 is pulled, by the first and second needle capture zones 116, 118, through the tissue and/or mesh. At block 910, the first and second needles 702, 704 are retracted to disengage the first and second needles 702, 704 from the respective first and second needle capture zones 116, 118. The method may further include retaining, by a retention device 120, the surgical fastener 102 in the tissue at block 912. These aspects are depicted at least in part in conjunction with the description of FIGS. 10-11 below.

FIG. 10 depicts a plurality of perspective views of installation of a surgical fastener, in accordance with the teachings of the present disclosure. In particular, FIG. 10 depicts the perspective view 1000 before the fastener deployment device 602 installs the surgical fastener 102, the perspective view 1020 depicts the fastener deployment device 602 having the needles 702, 704 fully extended and the surgical fastener 102 installed through the mesh 1002 and into the tissue 1004, and the perspective view 1040 depicts the surgical fastener 102 installed and the needles 702, 704 retracted from the surgical fastener 102. FIG. 11 depicts a perspective view and a side-view of an installed surgical fastener, in accordance with the teachings of the present disclosure. In particular, FIG. 11 depicts the perspective view 1100 and the side view 1150 of the surgical fastener 102 installed through the mesh 1002 and into the tissue 1004.

Returning to the description of the method 900 in conjunction with the descriptions of FIGS. 10 and 11, at block 908, the surgical fastener 102 is pulled, by the first and second needle capture zones 116, 118, through the tissue 1004. As depicted in FIGS. 10 and 11, the surgical fastener 102 first penetrates a mesh 1002 to affix the mesh 1002 to the tissue 1004. Multiple surgical fasteners 102 may be applied at different locations of the mesh 1002 to affix different portions of the mesh 1002 to different portions of the tissue 1004.

In the view 1000, before installation of the surgical fastener 102, a distal tip 1006 of the fastener deployment device 602 is positioned against the mesh 1002. The needle interface 604 is in a fully retracted position and is enclosed within the outer sleeve 614. Similar to the view 800 of FIG. 8, the surgical fastener 102 is loaded to the needle interface 604 (block 902), the first and second needles 702, 704 are extended along the respective first and second helical arms 110, 112 (block 904), and the first and second needles 702, 704 are engaged with the respective first and second needle capture zones 116, 118 (block 906). In some embodiments, the distal tip 1006 further includes tines (e.g., tines 716 of FIG. 7) to affix the mesh 1002 to the tissue 1004 before the surgical fastener 102 is installed.

In the view 1020, the surgical fastener 102 is pulled through the mesh 1002 and the tissue 1004 by the first and second needle capture zones 116, 118 (block 908). For example, block 908 may be performed responsive to a drive mechanism 616 extending the needles 702, 704, by way of the drive shaft 610, both axially along and radially around the longitudinal axis 114. When the needles 702, 704 extend, they extend out past the enclosure of the outer sleeve 614. As discussed throughout, the piercing—or penetrating—of the tissue 1004 may be performed by tips 710, 712 of the needles 702, 704 extending through the needle capture zones 116, 118, or may be performed by piercing tips 404 of the surgical fastener 102 itself when the needle capture zones 116, 118 fully enclose the tips 710, 712 of the needles 702, 704.

After the surgical fastener 102 is installed in the mesh 1002 and the tissue 1004, the needle interface 604 may retract back into the outer sleeve 614. When retracting, the needles may reverse over the path they traveled when installing the surgical fastener 102. As such, the needles 702, 704 will ride along the respective arms 110, 112 of the installed surgical fastener 102. As the needles 702, 704 retract, they disengage from the respective needle capture zones 116, 118 (block 910).

In some embodiments, the disengagement of the needles 702, 704 from the respective needle capture zones 116, 118 is aided by the retention of the surgical fastener 102 within the tissue 1004. The friction between the surgical fastener 102 and the tissue 1004 may provide enough of a retention force along the surface area of the helical arms 110, 112. However, in some embodiments, the retention may be increased by incorporating the retention devices 120 along one or both of the arms 110, 112 (block 912).

As depicted in the views 1100 and 1150 of FIG. 11, the surgical fastener 102 is installed through the mesh 1002 and into the tissue 1004. The base 104 abuts against the mesh 1002, and the retention devices 120 along the first and second arms 110 help retain the surgical fastener within the tissue 1004.

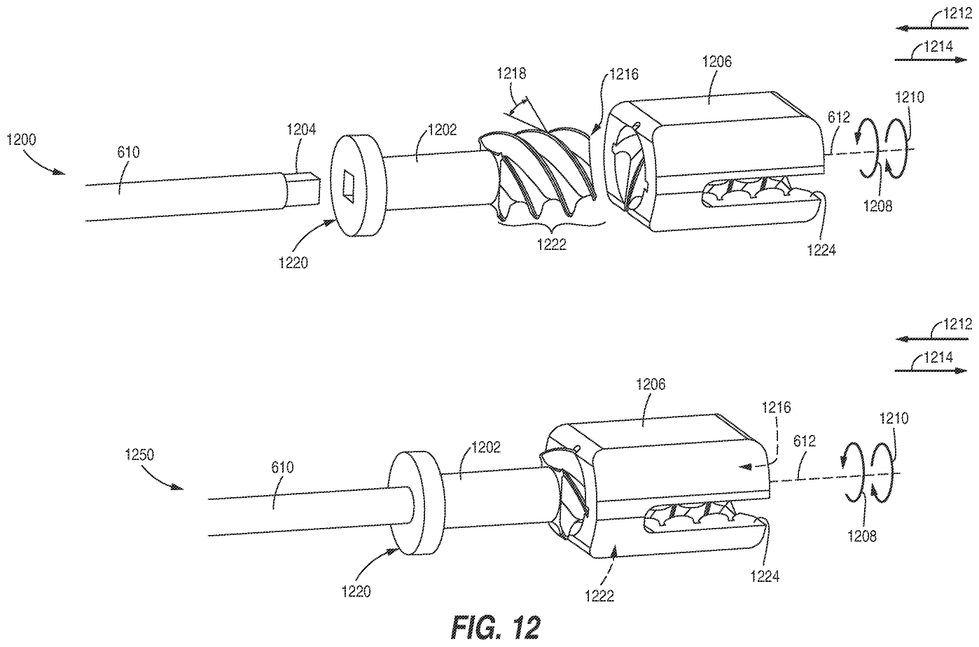

FIG. 12 depicts an exploded view and an assembled view of components of a fastener deployment device, in accordance with the teachings of the present disclosure. In particular, FIG. 12 depicts the perspective view 1200 of unassembled components of a fastener deployment device 602 and the perspective view 1250 depicts an assembled view of the components of the fastener deployment device 602. In particular, these components may be aspects of the drive mechanism 616.

In the views 1200 and 1250, the drive shaft 610 includes a proximal end 1204. The lead screw 1202 includes a distal end 1220 and a proximal end 1216. A portion of an outer circumference of the lead screw 1202 includes a threaded portion 1222. The threads have a pitch 1218, which corresponds to the pitch 402 of the surgical fastener 102.

A guide nut 1206 is fixedly mounted within a housing 622 (not depicted in FIG. 12), and the lead screw 1202 is configured to rotate through the guide nut 1206 in a first direction 1208 to extend the first and second spiral needles 702, 704, by way of the drive shaft 610, in the extension, or advancement, direction of 1212.

As shown in the assembled perspective view 1250, the proximal end 1204 of the drive shaft 610 is operatively coupled to the distal end 1220 of the lead screw 1202. The drive shaft 610 extends along the longitudinal axis 612 to the distal end 606 of the fastener deployment device 602, having the needle interface 604. When the lead screw 1202 rotates within the guide nut 1206 in a first direction 1208, the lead screw simultaneously translates in the extension, or advancement, direction 1212 to extend the needle interface 604. This translation and rotation motion is used to install a surgical fastener 102, (e.g., block 908 disclosing the pulling of the surgical fastener 102 through the tissue). Conversely, when the lead screw 1202 rotates within the guide nut 1206 in a second direction 1210, the second direction 1210 being opposite the first direction 1208, the lead screw 1202 translates in a retraction direction 1214 to retract the needle interface 604. This motion is used to disengage the needles 702, 704 from the needle capture zones, as disclosed herein (e.g., block 910).

The lead screw 1202 may be configured to rotate in the first direction 1208 responsive to an actuation force being applied to the proximal end 1216 of the lead screw 1202. Because the pitch 1218 of the lead screw 1202 corresponds to the pitch 402, the needles 702, 704 extend along the arms 110, 112 of the surgical fastener 102.

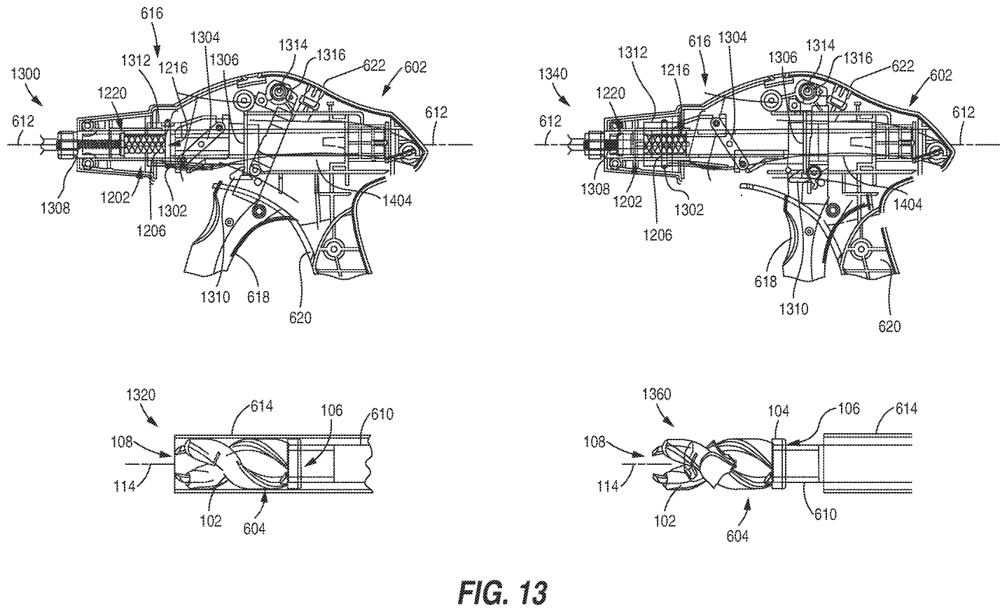

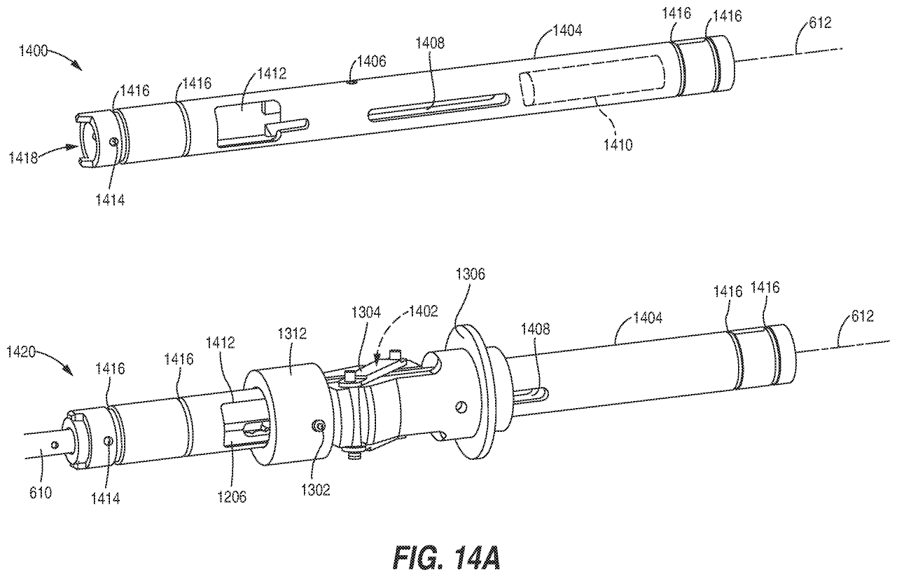

In some embodiments, the guide nut 1206 includes a crossbar slot 1224 configured to receive a crossbar (e.g., crossbar 1302 of FIG. 13) applying the actuation force to the proximal end 1216 of the lead screw 1202. The crossbar slot 1224 permits the axial travel of the crossbar 1302 into the guide nut 1206 to further reduce an axial length of the housing 622. In other embodiments, the actuation force is applied by a pin, or other object, to the proximal end 1216 of the lead screw 1202.

FIG. 13 depicts cross-sectional views of a drive mechanism and a needle interface in a retracted and an extended state, in accordance with the teachings of the present disclosure. In particular, on the left are the side views 1300 and 1320 depicting the drive mechanism 616 and the needle interface 604, respectively, when the fastener deployment device 602 is in a retracted state. On the right are the side views 1340 and 1360 depicting the drive mechanism 616 and the needle interface 604, respectively, when the fastener deployment device 602 is in an extended state.

As depicted in the views 1300 and 1340, the trigger 618 is operatively coupled to a slider 1306 via a trigger pawl 1310. In some of the embodiments disclosed herein, the slider 1306 may be realized by an annular, or circular, disc such that the trigger pawl 1310 may engage at any point along a circumference of the annular, or circular, slider. This permits rotation of aspects of the drive mechanism 616 about the longitudinal axis 612 relative to the housing 622. This feature allows for rotation of an articulation angle, discussed in more detail below.

In the various embodiments disclosed below, the drive mechanism 616 may vary the way the actuation force is applied. In a manual hand-trigger embodiment, the amount of retraction and extension of the needles 702, 704 is proportional to the amount of displacement of the trigger 618. In a stored-energy (e.g., impact-drive) embodiment, displacement of the trigger 618 translates the slider 1502 to store impact-drive energy in an impact drive spring. The impact drive spring transfers the stored impact-drive energy to accelerate the slider 1502. The accelerated slider 1502 applies the actuation force to a proximal end 1216 of the lead screw 1202. In a continuous-drive embodiment, displacement of the trigger 618 translates a slider 1502 in the housing 622 to store continuous-drive energy in a continuous drive spring. The continuous drive spring applies the actuation force to the proximal end 1216 of the lead screw 1202 during the advancement of the lead screw 1202. In yet another embodiment, the drive mechanism 616 includes an electric motor configured to extend both the first and second spiral needles axially along and radially around the longitudinal axis 114 simultaneously.

One example of a manual hand-triggered embodiment is depicted in the views 1300 and 1340. In such an embodiment, the trigger 618 is squeezed towards the handle 620. A trigger pawl 1310 is operatively coupled to the slider 1306 and causes the slider 1306 to translate to the right in the views 1300 and 1340. A reversing drive mechanism 1304, or a reversing link 1304, reverses the rightward motion of the slider 1306 to apply a leftward motion to the crossbar slider 1312 which contains the crossbar 1302. The leftward motion of the crossbar 1302 acts as the actuation force on the proximal end 1216 of the lead screw 1202.

The trigger 618 pivots about the trigger pivot 1314. A trigger torsion spring 1316 biases the trigger 618 towards the distal end 606 of the fastener deployment device 602. When a user displaces the trigger 618 towards the handle 620, energy is stored in the trigger torsion spring 1316. Upon release of pressure on the trigger 618, the energy stored in the trigger torsion spring 1316 biases the slider 1306 and the trigger 618 in the distal direction. Through the reversing link 1304, the crossbar slider 1312 is responsively translated in the proximal direction, and removes any force on the proximal end 1216 of the lead screw 1202.

As depicted in the views 1320 and 1360 of FIG. 13, the needle interface 604 transitions from the retracted state depicted in the view 1320 to the extended state depicted in the view 1360 in response to the lead screw 1202 being translated to the left as shown in FIG. 13. Initially the surgical fastener 102 is enclosed within the outer sleeve 614 in the retracted state view 1320 and extended (axially and rotationally) out of the outer sleeve 614 in the extended state view 1360. The view 1320 depicts the state of the needle interface 604 based on the state of the drive mechanism 616 depicted in the view 1300, and the view 1360 depicts the state of the needle interface 604 based on the state of the drive mechanism 616 depicted in the view 1340.

In some embodiments, the drive mechanism 616 further includes the lead-screw return spring 1308. During lead screw 1202 advancement along the longitudinal axis 612, and extension of the needle interface 604 along the longitudinal axis 114, the distal end 1220 of the lead screw 1202 compresses the lead-screw return spring 1308. The force applied to the trigger 618, is transferred to the slider 1306, reversed by the reversing drive mechanism 1304, and transmitted through the lead screw 1202 to provide the actuation force to compress the lead-screw return spring 1308. Upon a reduction in force applied to the trigger 618, the compressed lead-screw return spring 1308 biases the distal end 1220 of the lead screw 1202 in the retraction direction 1214. As the actuation force is removed from the proximal end 1216 of the lead screw 1202 by the trigger torsion spring 1316 restoring the slider 1306 and the trigger 618 to the initial position, the lead-screw return spring force restores the lead screw 1202 to an initial position (such as depicted in the view 1300), while simultaneously retracting the needle interface 604.

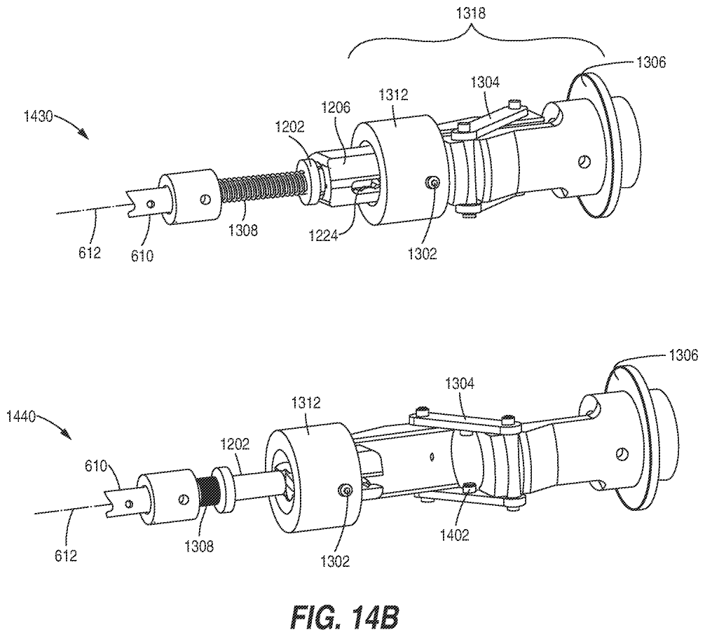

FIG. 14A depicts perspective views of a yoke and a yoke installed with aspects of the drive mechanism, in accordance with the teachings of the present disclosure. In particular, FIG. 14A depicts the perspective view 1400 of the yoke 1404. FIG. 14B depicts the perspective view 1420 of the yoke 1404 installed with components of the drive mechanism. The yoke 1404 extends along the longitudinal axis 612, and is free to rotate about the longitudinal axis 612 when installed within the housing 622. In the perspective view 1400 of the yoke 1404 alone, it can be seen that the yoke 1404 includes a reversing link pivot 1406, a slider slot 1408, a spring enclosure 1410, a guide nut receiving slot 1412, a roticulation connection point 1414, a plurality of circumferential grooves 1416, and a distal opening 1418.

As seen in the view 1420, aspects of the drive mechanism are assembled with the yoke 1404. A guide nut 1206 is able to be installed through the guide nut receiving slot 1412 transverse to the longitudinal axis 612. The guide nut 1206 is fixedly mounted within the yoke 1404 such that it does not translate along the longitudinal axis 612. Further, both the guide nut 1206 and the yoke 1404 rotate in tandem about the longitudinal axis 612. A lead screw 1202 (not depicted in the views of FIG. 14) operatively coupled to the drive shaft 610 may be inserted through the distal opening 1418 and threaded into the guide nut 1206. The reversing link 1304 may include a fixed pivot point 1402 that mates with the reversing link pivot 1406 on the yoke 1404. The slider 1306 translates along an outer surface of the yoke 1404 and is constrained by a pin on the slider 1306 being inserted into the slider slot 1408. The plurality of circumferential grooves 1426 mate with complimentary slots within the housing 622 to prevent the yoke 1404 from translating while permitting the yoke 1404 to rotate within the housing 622. Further, a groove in the plurality of circumferential grooves 1426 may have a clip inserted into the groove to longitudinally constrain a spring disposed on an outside circumferential surface of the yoke 1404.

FIG. 14B depicts perspective views of components of a drive mechanism in a retracted and an extended state, in accordance with the teachings of the present disclosure. In particular, FIG. 14B depicts the perspective view 1430 on the top and the view 1440 on the bottom of a drive mechanism 616 that may be used in the hand-triggered embodiment depicted in FIG. 13. The view 1430 depicts the components in a retracted state (e.g., similar to that of the view 1300) and the view 1440 depicts the components in an extended state (e.g., similar to that of the view 1340) separated from the housing 622.

The drive mechanism may include a reversing drive mechanism 1318 that includes a slider 1306, a reversing link 1304, and a crossbar slider 1312. The slider 1306 may be realized by an annular disc. Not depicted in the views 1430 and 1440 is the trigger pawl 1310, which may be operatively coupled to the outer-circumference of the slider 1306. The reversing link 1304 converts the rightward movement of the slider 1306 into a leftward movement of the crossbar slider 1312 which contains the crossbar 1302 by pivoting about the fixed pivot point 1402 (shown in the view 1440). As the crossbar 1302 moves leftward, it applies the actuation force to the proximal end of the lead screw 1202, causing the lead screw 1202 to rotate about the longitudinal axis 612 and translate to the left. The lead-screw return spring 1308 is not compressed in the view 1430 (e.g., is not biasing the lead screw 1202 in the right-ward direction) and is compressed in the view 1440 (e.g., is biasing the lead screw 1202 in the right-ward direction).

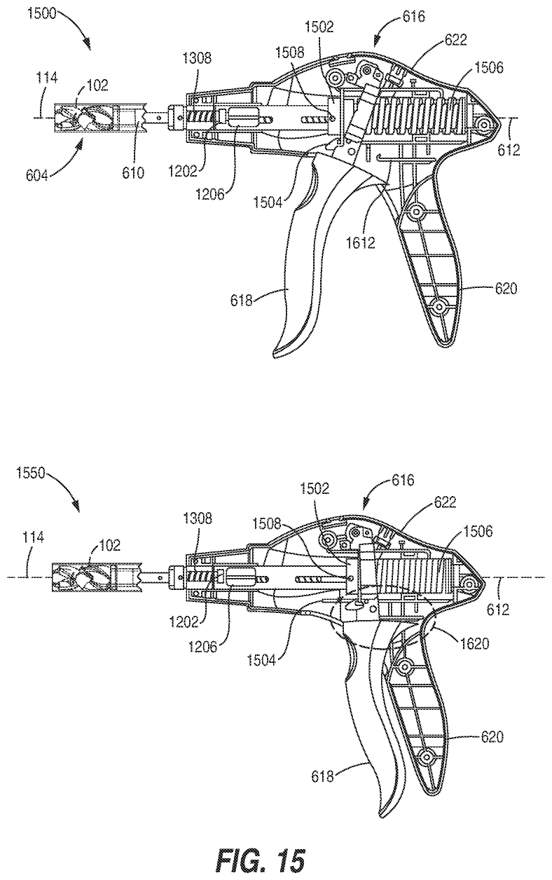

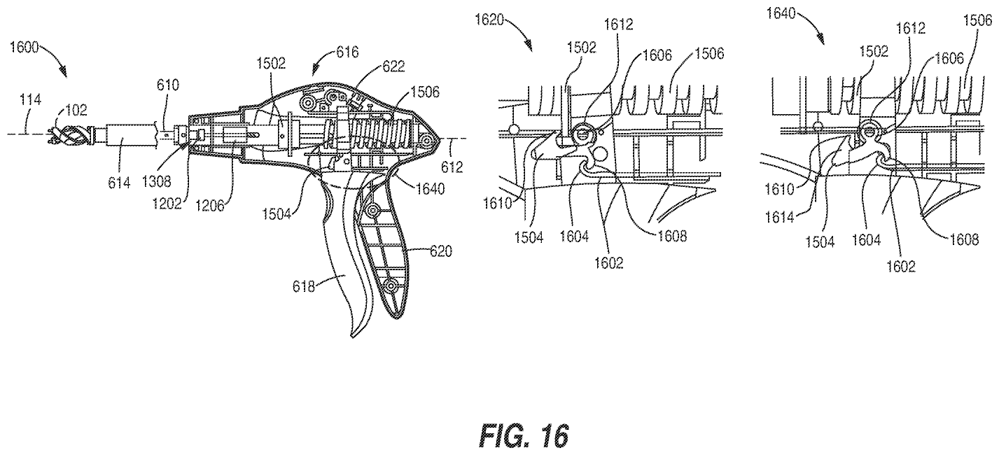

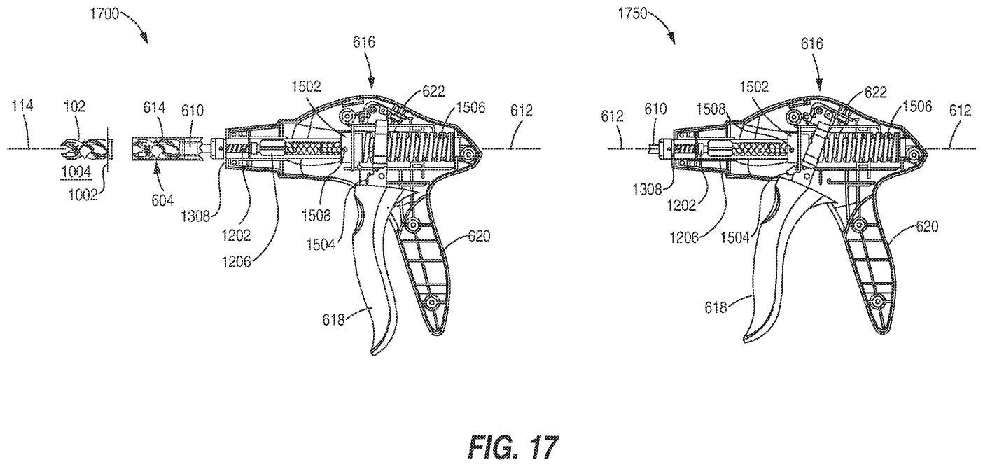

FIGS. 15-17 depict cross-sectional views of an impulse energy drive mechanism, in accordance with the teachings of the present disclosure. In particular, FIG. 15 depicts the cross-sectional view 1500 on the left and the cross-sectional view 1550 on the right. The views 1500 and 1550 depict a drive mechanism 616 that may be realized as a stored-energy actuation device. FIG. 16 depicts the views 1600, 1620, and 1640 during stored energy release. FIG. 17 depicts the cross-sectional views 1700 and 1750 of the impulse energy drive mechanism after installation of a surgical fastener, in accordance with the teachings of the present disclosure. FIGS. 15-17 will be discussed together along with the aspects of installing the surgical fastener 102 per the method 900.

The views of FIG. 15-17 include aspects of the impulse energy drive mechanism 616. Similar to the drive mechanism of FIGS. 13-14, the trigger 618 is configured to be squeezed towards the handle 620 to initiate the installation of the surgical fastener 102. Here, instead of proportional displacement of the trigger 618 resulting in proportional extension/retraction of the needle interface 604 like in the drive mechanism 616 of FIGS. 13-14, the displacement of the trigger 618 towards the handle 620 results in the slider 1502, operatively coupled to the trigger 618 via the trigger pawl 1504, to translate to the right to compress (e.g., store energy) in the impact drive spring 1506. The impact drive spring 1506 may be disposed within the spring enclosure 1410 of the yoke 1404. In other embodiments, the impact drive spring 1506, or other similar spring of a drive mechanism, may be disposed on an outside circumference of the yoke 1404, such as depicted in FIG. 17. Once the trigger 618 is fully displaced, as depicted in the view 1550, the trigger 618 is disengaged from the slider 1502, and the stored energy in the impact drive spring 1506 accelerates the slider 1502 towards the lead screw 1202. Similar to the embodiment disclosed above, a threaded portion 1222 of the lead screw 1202 is inserted within the fixedly mounted guide nut 1206. The actuation force is realized from the accelerated slider 1502 impacting the proximal end of the lead screw 1202 via a cross bar 1508 that is contained in the slider 1502. As discussed more fully below, the impact drive spring 1506 may be disengaged from the slider 1502 and no longer applying a force to the slider 1502. The actuation of the impulse energy drive mechanism is discussed in more detail below.

As depicted in the view 1500, the surgical fastener 102 is loaded to the needle interface 604, with the needles 702, 704 extending along the arms 110, 112 and being engaged with the needle capture zones 116, 118 (blocks 902, 904, and 906 of the method 900). The drive mechanism 616 includes the slider 1502, which is similar to the slider 1306 of FIGS. 13 and 14 except that the slider 1502 contains a cross bar 1508 that acts on the proximal end 1216 of the lead screw 1202. Here, the slider 1502 is selectively engaged to the trigger 618 via the trigger pawl 1504. When the trigger pawl 1504 is engaged to the slider 1502, displacement of the trigger 618 towards the handle 620 causes a translation of the slider 1502 to the right along the longitudinal axis 612 as depicted in the view 1500.

As seen in the view 1550 of FIG. 15, this rightward translation of the slider 1502 compresses the impact drive spring 1506 to store the impact-drive energy. In the view 1550, the trigger 618 is nearly fully displaced towards the handle 620, and the impact drive spring 1506 is compressed and storing the impact-drive energy. The lead screw 1202, as well as the needle interface 604 within the outer sleeve 614, remains stationary between the views 1500 and 1550.

The close-up cross-sectional views 1620 and 1640 depicts aspects of the slider 1502, the trigger pawl 1504, the impact drive spring 1506, and the trigger pawl clip 1602 before the trigger pawl 1504 is released from the slider 1502 (view 1620) and after the trigger pawl 1504 is released from the slider 1502 (view 1640). The view 1620, depicting a close-up from the view 1600, shows the trigger pawl 1504 engaged via its latch 1610 to the slider 1502. The trigger pawl 1504 is able to rotate around a pivot point 1606, and a torsion spring 1612 holds the trigger pawl 1504 in a clockwise/upward biasing rotation towards the slider 1502 to maintain hold of the latch 1610 of the trigger pawl 1504 upon the slider 1502. In view 1620, the user has pulled the trigger 618 fully towards the handle 620, causing the trigger pawl clip 1602 to deflect downwards so that the hook 1604 of the trigger pawl clip 1602 engages with the tail 1608 of the trigger pawl 1504. As the user begins to release pressure on the trigger 618, the trigger begins to rotate distally towards its starting position (e.g., biased by the trigger torsion spring 1316), and the trigger pawl 1504 which is attached to the trigger 618 also begins to move distally. The trigger pawl clip 1602 (which is fixedly attached to the inside of the housing 622) stays stationary. This distal movement of the trigger pawl 1504 (towards the left in FIG. 16) causes the hook 1604 of the trigger pawl clip 1602 to pull on the tail 1608 of the trigger pawl 1504. This pull overcomes the force of the torsion spring 1612 and causes rotation of the trigger pawl 1504 (in a downward/counter-clockwise direction in FIG. 16) about pivot point 1606. This causes the latch 1610 of the trigger pawl 1504 to disengage from the slider 1502.

Although this configuration causes the trigger pawl 1504 to release the slider 1502 upon the start of release of the trigger 618 after it has reached its most compressed state, the components could alternatively be configured to cause the trigger pawl 1504 to pivot and release the slider 1502 at the initial point when the trigger 618 reaches its most compressed position toward the handle 620.

As seen in the view 1640, when the trigger pawl 1504 pivots, the latch 1610 disengages from the slider 1502. The impact drive spring 1506 releases the stored impact-drive energy by expanding and accelerating the slider 1502 distally towards the lead screw 1202. After full expansion of the impact drive spring 1506, the slider 1502 separates from the impact drive spring 1506, and the impact drive spring 1506 no longer transfers its stored impact-drive energy to the slider 1502. A momentum of the accelerated slider 1502 applies the actuation force to the proximal end of the lead screw 1202. In some embodiments, the slider 1502 includes a cross bar 1508 similar to the crossbar 1302 to apply the actuation force.

As seen in the view 1600, when the lead screw 1202 receives the actuation force from the slider 1502, the lead screw 1202 rotates about the longitudinal axis 612 within the guide nut 1206 and simultaneously translates leftwards along the longitudinal axis 612. The needle interface 604, operatively coupled to the lead screw 1202 via the drive shaft 610, is also extended and rotated outside of the outer sleeve 614 to install the surgical fastener 102 (block 908 of method 900).

The lead screw 1202 compresses the lead-screw return spring 1308, which biases the lead screw 1202 rightwards. Because the momentum of the accelerated slider 1502 has dissipated (e.g., by compressing the lead-screw return spring, by friction of the surgical fastener 102 being installed in the tissue) and the impact drive spring 1506 is separated from, and not applying a left-wards force to the slider 1502, the lead screw 1202 is translated rightward, such as depicted in the view 1700. As the lead screw 1202 translates rightward, the needle interface 604 is also retracted, which causes the needles to disengage from the needle capture zones (block 910 of method 900). In the view 1700, the surgical fastener 102 has been installed through the mesh 1002 and into the tissue 1004, the needle interface 604 has retracted to be enclosed within the outer sleeve 614, and the lead-screw return spring 1308 has extended to bias the lead screw 1202 and the slider 1502 rightwards to the initial starting positions they were at in the view 1500.

In the view 1700, the trigger is still displaced towards the handle 620. However, as depicted in the view 1750, when the trigger 618 is restored to the position it was in the view 1500 (e.g., an initial starting position), the trigger pawl 1504 reengages with the slider 1502. To achieve this reengagement, as the trigger pawl 1504 approaches the slider 1502, the ramped leading edge 1614 of the trigger pawl interacts with the annular disc on the slider 1502, causing the trigger pawl 1504 to rotate (counter clock-wise/downward in view 1700) about the pivot point 1606, overcoming the force of the torsion spring 1612. After the latch 1610 of the trigger pawl 1504 has traveled past the annular disc of the slider 1502, the torsion spring 1612 rotates (upward/clockwise in view 1750) the trigger pawl 1504, causing the trigger pawl latch 1610 to become reengaged with the annular disc of the slider 1502.

In the impulse-energy drive mechanism embodiments disclosed herein, the spring constants of the impact drive spring 1506 and the lead-screw return spring 1308 are selected to achieve proper installation of the surgical fastener 102. For example, the impact drive spring 1506 is selected to have a spring constant that is appropriate for a user's hand to compress the trigger 618 towards the handle 620. Further, the impact drive spring 1506 spring constant is selected to store sufficient energy to accelerate the slider 1502 to apply a sufficient actuation force to the proximal end of the lead screw 1202 to install the surgical fastener 102 into a tissue. The lead-screw return spring 1308 is selected to have a spring constant to absorb a sufficient amount of energy from the lead screw 1202 to charge the lead-screw return spring 1308 while maintaining a sufficient amount of energy imparted on the slider 1502 from the impact drive spring 1506 for the surgical fastener 102 to be pulled through the tissue by the needle capture zones 116, 118.

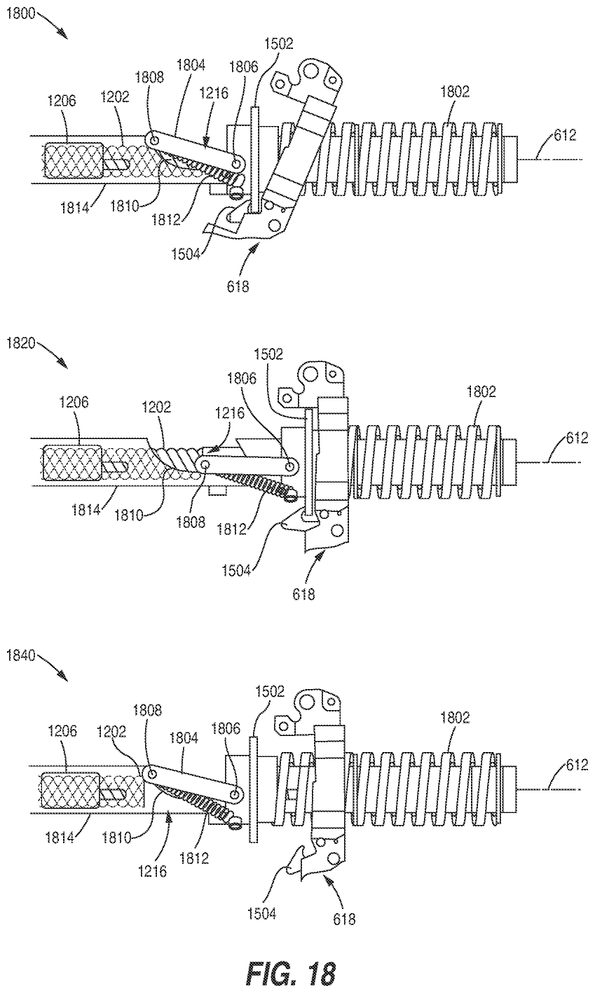

FIG. 18 depicts cross-sectional views of aspects of a continuous energy drive mechanism, in accordance with the teachings of the present disclosure. In particular, FIG. 18 depicts the view 1800 of aspects of a continuous energy drive mechanism at an initial state, the view 1820 of aspects of the continuous energy drive mechanism at a stored-energy state, and the view 1840 of aspects of the continuous energy drive mechanism at an energy-released state.

The views 1800, 1820, and 1840 depict the lead screw 1202 installed within a guide nut 1206 housed in a cylindrical yoke 1814. The cylindrical yoke 1814 may be similar to the yoke 1404 in that it includes a guide nut receiving slot, constrains the slider to translating along the longitudinal axis 612, and permits rotation of the yoke 1814 within the housing, but it further includes a ramped surface 1810 that serves to disengage the pivot pusher 1804 from the proximal end 1216 of the lead screw 1202 after installation of the surgical fastener 102. A trigger 618 selectively engages the slider 1502 via the trigger pawl 1504. A pivot pusher 1804 is mechanically connected at a pivot point 1806 to the slider 1502. A pivot spring 1812 biases the crossbar 1808 of the pivot pusher 1804 towards a ramped surface 1810 of the yoke 1814.

In the initial state of the view 1800, the lead screw 1202 is fully retracted to the right direction, and the crossbar 1808 rests on a side surface of the lead screw 1202. The pivot spring 1812 is biasing the crossbar 1808 towards the ramped surface 1810, but the lead screw 1202 is in a retracted position and does not permit the pivot pusher 1804 to rotate counter-clockwise, as depicted in the view 1800. The trigger 618 is not displaced towards a handle 620 (not depicted in FIG. 18), but the trigger 618 is engaged to the slider 1502 via the trigger pawl 1504.

The continuous energy drive spring 1802 may have reserve stored energy when in its fully extended state (e.g., as seen in the view 1840). This additional reserve energy, realized as a pre-load on the continuous energy drive spring 1802, ensures that the needle interface 604 has sufficient energy to pull the surgical fastener 102 through the mesh and/or tissue it is penetrating. Once the surgical fastener 102 is fully installed (e.g., the needle interface 604 has fully extended), the pivot pusher 1804 has pivoted away from the proximal end 1216 of the lead screw 1202, thus removing the force being applied from the continuous energy drive spring 1802. This is in contrast to a stored-energy embodiment disclosed above which generally has the impact drive spring 1506 without a preload.

In the stored-energy state depicted in the view 1820, the trigger 618 has been displaced towards the handle 620. Because the trigger 618 is engaged, via the trigger pawl 1504 to the slider 1502, the slider 1502 translates along the yoke 1814 to the right along the longitudinal axis 612. This translation compresses the continuous energy drive spring 1802 to build up additional potential energy in the continuous energy drive spring 1802. Also, as the slider 1502 translates to the right, the pivot pusher 1804 is also translated to the right. As the crossbar 1808 slides across the outer circumference of the lead screw 1202, its angle relative to the longitudinal axis 612 remains constant until the pivot pusher 1804 has been sufficiently retracted with the crossbar 1808 being pulled further to the right past the proximal end 1216 of the lead screw 1202.

The view 1820 depicts the stored-energy state after the slider 1502 has been translated to the right and the pivot pusher 1804 has pivoted down, and is now parallel with the longitudinal axis 612 after being biased by the pivot spring 1812, to interface with the proximal end 1216 of the lead screw 1202. Similar to the impact drive mechanism of the FIGS. 13-15, the trigger 618 is selectively engaged with the slider 1502 by the trigger pawl 1504.

As the trigger 618 is further compressed, the trigger pawl 1504 is disengaged from the slider 1502, as depicted in the view 1840. Here, the energy stored in the continuous energy drive spring 1802 applies the actuation force to the proximal end 1216 of the lead screw 1202, through the pivot pusher 1804 to insert the surgical fastener 102 into a tissue and/or mesh. This is distinguished from the impact drive mechanism of the FIGS. 13-15 that uses momentum from an accelerated slider 1502 to apply the actuation force. As the continuous energy drive spring 1802 expands and releases its stored energy, the slider 1502 is driven leftward. Because the crossbar 1808 of the pivot pusher 1804 is engaged with the proximal end 1216 of the lead screw 1202, this actuation force is translated to the needle interface 604 (not depicted) to pull the surgical fastener 102 into the mesh and/or tissue (block 908 of the method 900). As the slider 1502 and the pivot pusher 1804 translate to the left, the crossbar 1808 interfaces with (e.g., rides along) the ramped surface 1810 of the yoke 1814, and is pivoted upwards to disengage the crossbar 1808 from the proximal end 1216 of the lead screw 1202, as depicted in the view 1840.

The lead screw 1202 may also compress a lead screw return spring (e.g., similar to the lead-screw return spring 1308) as it extends the needle interface 604. Thus, as the continuous energy drive spring 1802 expands, its energy is dissipated by both the friction from installing the surgical fastener 102 and compressing the lead-screw return spring 1308. However, after the pivot pusher 1804 has pivoted upwards and is disengaged from applying the actuation force to the proximal end 1216 of the lead screw 1202, the continuous energy drive spring 1802 no longer applies a force to the lead screw 1202. The length, along the longitudinal axis 612, of the ramped surface 1810 may correspond to a desired insertion length of the surgical fastener 102. Thus, after the surgical fastener 102 is fully installed with its base 104 abutting against the mesh and/or tissue, the crossbar 1808 is disengaged from the proximal end 1216 of the lead screw 1202 by the ramped surface 1810.

The pivot pusher 1804 may maintain its pivoted position (depicted in the view 1840) because the continuous energy drive spring 1802 may prevent the slider 1502 from translating rightwards. After installation of the surgical fastener 102 and disengagement of the crossbar 1808 from the proximal end 1216 of the lead screw 1202, the lead-screw return spring 1308 biases the distal end of the lead screw 1202 right-wards towards the guide nut 1206 back to the initial state, thereby retracting the needle interface 604 (block 910 of the method 900). After the lead screw 1202 has returned to its initial state, the trigger 618 may also be returned to its initial state to reengage the trigger pawl 1504 to the slider 1502, thus returning the continuous energy drive mechanism to the initial state depicted in the view 1800. In some embodiments, the lead-screw return spring 1308 is pre-loaded at the initial starting position.

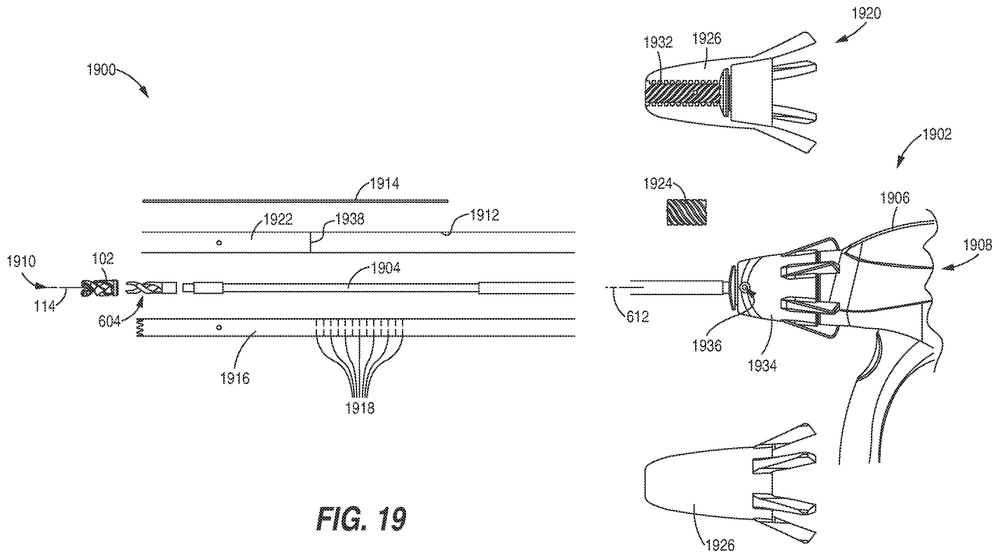

FIG. 19 depicts unassembled components of an articulation device, in accordance with the teachings of the present disclosure. In particular, FIG. 19 depicts an exploded side view 1900 of the fastener deployment device 1902. Similar to the fastener deployment device 602, the fastener deployment device 1902 includes a proximal end 1908 and a distal end 1910. The fastener deployment device 1902 includes a drive mechanism 1906, which may be realized by any of the drive mechanisms 616 described herein. The longitudinal axis 612 is depicted on the proximal end 1908 along the flexible drive shaft 1904. The longitudinal axis 114 is depicted along the surgical fastener 102.

When the fastener deployment device 1902 is in a straight position (e.g., not articulated), the longitudinal axis 114 may be coincident with the longitudinal axis 612. However, when the fastener deployment device 1902 is articulated, the longitudinal axis 114 will be at an angle with the longitudinal axis 612. A flexible drive shaft 1904, flexed at the articulation joints 1918, is configured to receive an insertion input from the drive mechanism 1906 at the proximal end 1908 (e.g., the extension and rotation of the lead screw within the drive mechanism 1906 about the longitudinal axis 612) and transmit the insertion input into a complimentary extension and rotation of the needle interface 604 to install the surgical fastener 102 about the longitudinal axis 114 at the distal end 1910. The flexible drive shaft 1904 may be similar to the drive shaft 610 disclosed herein, but is able to flex about an articulation joint.

The flexible drive shaft 1904 is installed within an inner flexible tube 1916 that includes a plurality of articulation joints 1918 along the length of the inner flexible tube 1916. The inner flexible tube 1916 is disposed around the flexible drive shaft 1904 and is disposed, at least partially, within the proximal outer sleeve 1912. An articulation tendon 1914 connects the distal end 1910 of the fastener deployment device 1902 to the proximal outer sleeve 1912.

The articulation device further includes an articulation actuator 1920 configured to retract the proximal outer sleeve away from the distal end 1910 to sequentially expose the articulation joints in the plurality of articulation joints 1918. The retraction of the proximal outer sleeve 1912 causes the distal end 1910, pulled by the articulation tendon 1914, to bend about the exposed articulation joints 1918.

The articulation actuator 1920 may include an articulation lead screw 1924 that fits inside an articulation turn knob 1926 containing threads 1932 that match those of the articulation lead screw 1924. Rotation of the articulation turn knob 1926 about the longitudinal axis 612 causes the proximal outer sleeve 1912, which is fixed to the articulation lead screw 1924, to retract towards the proximal end 1908. The proximal outer sleeve 1912 and the attached articulation lead screw 1924 do not rotate relative to axis 612. Therefore, as the articulation turn knob 1926 is turned, the lead screw is moved axially (not rotationally) along axis 612.

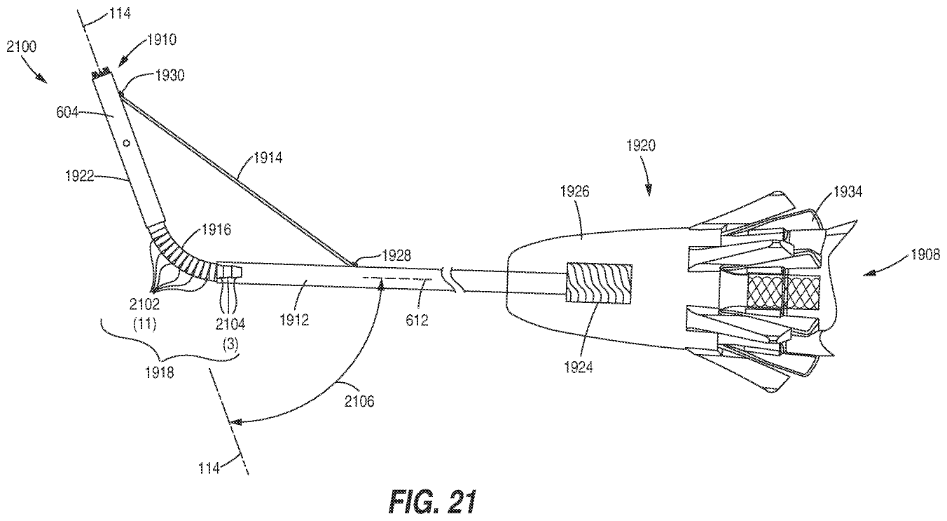

The articulation device may further include a roticulation turn knob 1934. The roticulation turn knob includes a yoke connection point 1936. The yoke connection point 1936 is configured to be mechanically coupled to the roticulation connection point 1414 of the yoke 1814 (or similar connection point of the yoke 1814 of a continuous energy embodiment). This mechanical coupling permits both the yoke 1814 and the distal end 1910 to rotate together about the longitudinal axis 612. In embodiments with a trigger pawl engaging an annular disk on a slider, the housing 622, trigger 618, and the handle 620 may remain stationary as rotation of the roticulation turn knob 1934 rotates both the distal end 1910 and the yoke 1814. When the distal end 1910 is articulated (e.g., the view 2100 of FIG. 21), the angle of insertion (e.g., longitudinal axis 114) may be changed relative to the orientation of the housing 622.

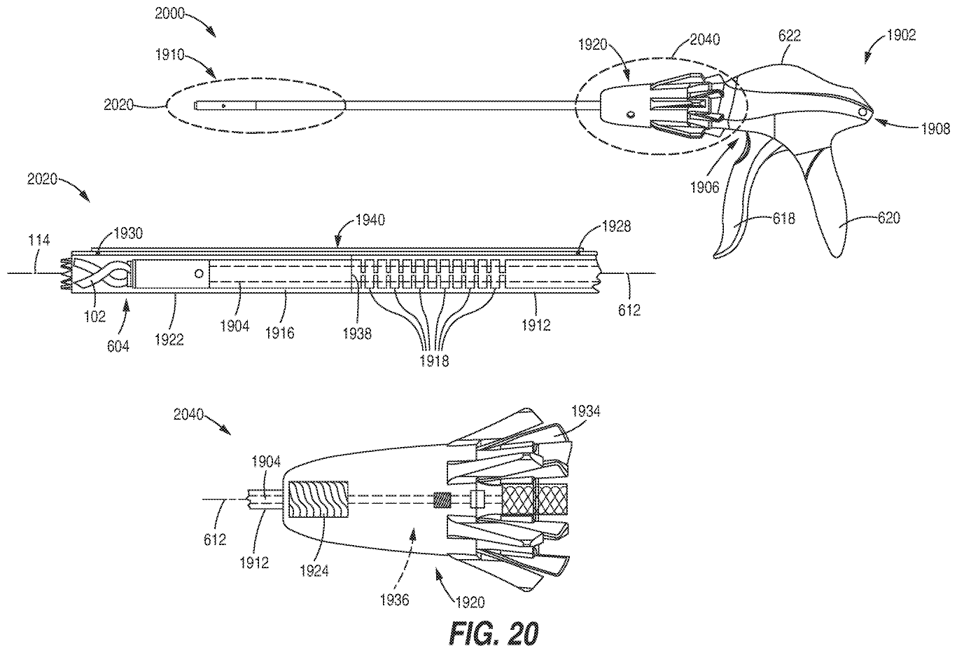

FIG. 20 depicts an assembled articulation device in a straight state, in accordance with the teachings of the present disclosure. In particular, FIG. 20 depicts the side view 2000 of the fastener deployment device 1902 in a straight state. The view 2020 is a close-up cross section of the distal end of the fastener deployment device 1902 and the view 2040 is a close-up cross section of the proximal end of the fastener deployment device 1902.

As seen in the view 2000, the fastener deployment device 1902 includes a housing 622, a trigger 618, a handle 620, a drive mechanism 1906, and an articulation actuator 1920, all disposed on the proximal end 1908. The fastener deployment device 1902 is in a straight state, and all of the articulation joints in the plurality of articulation joints are enclosed within the proximal outer sleeve 1912. The articulation tendon 1914 is connected to the proximal outer sleeve 1912 at the point 1928 and to the distal end 1910 at the point 1930.

In some embodiments, increased rigidity may be achieved by configuring the articulation joints 1918 to be recessed inside the proximal outer sleeve 1912 (e.g., translated proximally along the longitudinal axis 612) such that the most distal articulation joint 1918 is displaced from the distal end 1938 of the proximal outer sleeve 1912. Recessing the most distal articulation joint 1918 (e.g., locating all articulation joints 1918 proximally) requires additional retraction of the proximal outer sleeve 1912 before exposing the most distal articulation joint 1918. As such, the articulation tendon 1914 is connected to the proximal outer sleeve 1912 at the connection point 1928 that is realized as a sliding connection point. The connection point 1928 is permitted to slide longitudinally along the proximal outer sleeve 1912 a distance equal to the amount the most proximal articulation joint 1918 is recessed from the distal end 1938 of the proximal outer sleeve 1912. Alternatively, the connection 1930 on the distal end 1910 may longitudinally slide similar to the description of the sliding connection point 1928 discussed above.

The proximal outer sleeve 1912 is configured to slide along longitudinal axis 612 to sequentially cover and/or expose the articulation joints 1918 depending on the direction of translation of the proximal outer sleeve 1912. In some embodiments, the fastener deployment device 1902 includes a distal outer sleeve 1922 that is fixedly attached to the distal end 1910. In such an embodiment, the articulation tendon 1914 attaches to the distal outer sleeve 1922. The proximal outer sleeve 1912 may be a rigid proximal outer sleeve 1912 that prevents the bending of the articulation joints 1918 when the articulation joints 1918 are disposed within the proximal outer sleeve 1912.

The articulation tendon 1914 is disposed along the proximal outer sleeve 1912 and the distal end 1910 and may be under a slight tensile force between the points 1928 and 1930 to maintain the articulation tendon 1914 against the fastener deployment device 1902, as compared to loosely flapping away from the fastener deployment device 1902. The fastener deployment device may be configured to install a surgical fastener 102 when in a straight position as described in detail in conjunction with the fastener deployment device 602 herein. In such an embodiment, the surgical fastener 102 is installed into a tissue along the longitudinal axis 114 that is coincident with the longitudinal axis 612. However, it may be desired to install a surgical fastener 102 along a longitudinal axis 114 that is at an angle to the longitudinal axis 612. As such, the fastener deployment device 1902 may be transitioned to an articulated state, such as depicted in the view 2100 of FIG. 21.

FIG. 21 depicts an assembled articulation device in an articulated state, in accordance with the teachings of the present disclosure. In particular, FIG. 21 depicts the view 2100 of a distal end 1910 and the articulation actuator 1920 of the fastener deployment device 1902 when in an articulated state.

In the view 2100, the articulation turn knob 1926 has been rotated to retract the proximal outer sleeve 1912. The retraction of the proximal outer sleeve 1912 caused the articulation joints 2102 to be exposed, with the articulation joints 2104 remaining enclosed within the proximal outer sleeve 1912. Further, the retraction of the proximal outer sleeve 1912 increases a tension on the articulation tendon 1914. The tensile force of the articulation tendon 1914 causes the distal end 1910 to bend about the exposed articulation joints 2102.

Each of the articulation joints may be configured to bend a set articulation angle. For example, an inner flexible tube 1916 may have fourteen (14) articulation joints 1918, each articulation joint 1918 may be configured to bend six (6) degrees. Thus, when a first articulation joint (e.g., the articulation joint closest to the distal end 1910) is exposed by the retracting proximal outer sleeve 1912, the distal end 1910 bends six (6) degrees, providing a six (6) degree mismatch between the longitudinal axis 114 and the longitudinal axis 612 (e.g., as depicted by the articulation angle 2106). With each sequential articulation joint 1918 being exposed, the degree of mismatch (e.g., the amount of articulation) between the longitudinal axes 114, 612 is incrementally increased by five degrees.

As seen in the embodiment depicted in the view 2100, eleven (11) articulation joints 2102 are exposed and three (3) articulation joints 2104 are covered, of the total fourteen (14) articulation joints 1918. The degree of mismatch (e.g., the articulation angle 2106) between the longitudinal axis 114 and the longitudinal axis 612 is approximately 66 degrees. If each exposed articulation joint 2102 bends at the same set articulation angle, then six (6) degrees of bend is realized at each of the eleven exposed articulation joints 2102. Further rotation of the articulation turn knob 1926 would further retract the proximal outer sleeve 1912 and increase the tension on the articulation tendon 1914 to expose the next sequential articulation joint 2104 that is presently enclosed in the view 2100. As a result, the plurality of articulation joints 1918 would include twelve (12) exposed articulation joints 2102 and two (2) covered articulation joints 2104. The articulation angle 2106 would be incrementally increased by six (6) degrees to 72 degrees. The full retraction of the proximal outer sleeve 1912 to expose the remaining two covered articulation joints 2104 would provide for an additional twelve (12) degrees of articulation for a total of 84 degrees for the articulation angle 2106.

In other embodiments, the articulation bend angle of each articulation joint 1918 may vary between one (1) degree and twenty five (25) degrees, with a smaller articulation bend angle providing finer control of the articulation angle as compared to the larger articulation bend angles. In one such embodiment, the articulation joint(s) 1918 closest to the distal end 1910 include articulation joints 1918 that have a gross-adjustment articulation angle (e.g., 10-15 degrees) while the articulation joints 1918 sequentially closer to the proximal end 1908 have fine-adjustment articulation angles (e.g., 1-4 degrees). This permits an operator to more quickly obtain a desired articulation angle for the same amount of rotation input to the articulation turn knob 1926 as the first few turns of the articulation turn knob 1926 provide gross changes to a typical target articulation angle 2106, with subsequent turns of the articulation turn knob 1926 providing for fine control of the articulation angle 2106.

The fastener deployment device 1902 may be transitioned from the articulated state (of view 2100) back to the straight state (of view 2000) by rotating the articulation turn knob 1926 in an opposite direction to extend the proximal outer sleeve 1912 towards the distal end 1910. The opposite rotation of the articulation turn knob 1926 extends the proximal outer sleeve 1912 towards the distal end 1910 which simultaneously covers the exposed articulation joints 2102 and reduces a tension on the articulation tendon 1914. This causes the distal end 1910 to straighten with each sequential articulation joint 1918 that is covered.

In some embodiments, the articulation tendon 1914 consists of only one articulation tendon 1914 that applies and releases a tensile force on the distal end 1910. A second articulation tendon, for example a second articulation tendon disposed on an opposite side of the proximal outer sleeve 1912 as the first articulation tendon to apply a counter-tensile force to straighten the distal end 1910, is not needed. The second articulation tendon is not needed because the extension of the proximal outer sleeve 1912 provides the means for restoring the distal end 1910 to the straight position. In addition, with the articulation configuration described, each exposed articulation joint 2102 bends to a maximum articulation angle as it is exposed from the proximal outer sleeve 1912. Therefore, the segments of the joints are not able to over-flex in this condition. The causes the articulation angle to be held in a rigid state that is able to resist the potential over-flexion that would be caused by pushing the distal end 1910 of the fastener deployment device against the fastening interface. This configuration could prevent the need for a second tendon along the outer aspect of the articulation that would oppose over-flexion.

As the articulation device is configured to bend the distal end in a single direction, rotation of the distal end about the longitudinal axis 612 may be desired to achieve a desired surgical fastener 102 angle of installation (e.g., a desired orientation of the longitudinal axis 114). Thus, drive mechanisms 616 that are free to rotate relative to the housing 622, and the trigger 618, may be desired. Turning of the roticulation turn knob 1934 may be used to achieve this rotation as it is attached to drive mechanisms 616 through the yoke. As disclosed above, the trigger pawls engaging the sliders on a circular, or annular, disc provides for this desired rotation. During roticulation, the articulated distal end 1910 is able to sweep through all quadrants (e.g., may sweep into or out of the page as depicted in the view 2100).

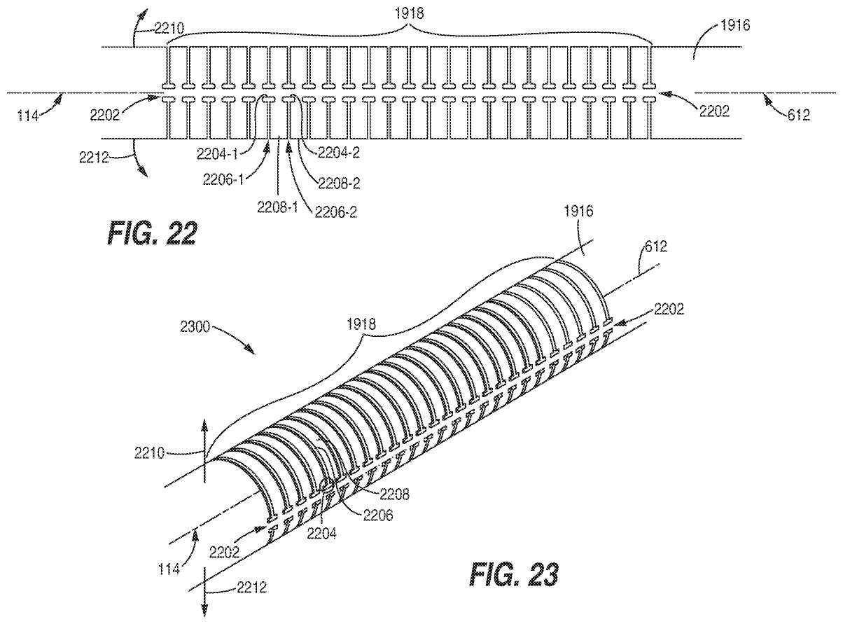

FIG. 22 depicts a side view of an exemplary inner flexible tube, in accordance with accordance with the teachings of the present disclosure. FIG. 23 depicts a perspective view of an exemplary inner flexible tube, in accordance with the teachings of the present disclosure. In particular, FIG. 22 depicts the side view 2200 of the inner flexible tube 1916 and FIG. 23 depicts the perspective view 2300 of the inner flexible tube 1916.

The inner flexible tube 1916 is oriented in the views 2200 and 2300 with the proximal end on the right (with longitudinal axis 612 extending right-wards) and the distal end on the left (with the longitudinal axis 114 extending left-wards). The inner flexible tube may be manufactured as a flexible stainless steel component able to be bent in either an upwards direction 2210, a downwards direction 2212, or both directions 2210, 2212. The inner flexible tube 1916 resists bending perpendicular (e.g., to the left and to the right) to the upward 2210 and downward 2212 directions.

In the views 2200 and 2300, the articulation joints 1918 are formed by providing gaps 2206 on either side of the segments 2208. The gaps 2206 form semi-circles that transverse the longitudinal axes 612, 114, but do not go all the way through the inner flexible tube 1916, leaving a stiffener column 2202 on both sides of the gaps 2206. The gaps 2206 may be terminated with expansion slots 2204 that permit easier bending of each articulation joint 1918.

As the inner flexible tube 1916 is articulated, the gaps 2206 on the side the inner flexible tube 1916 is being articulated towards are closed and the opposite side gaps 2206 are opened. Each articulation joint 1918 bends at its maximum articulation angle when the gap 2206 is fully closed (e.g., adjacent segments 2208-1, 2208-2 abut against each other). In general, the inner flexible tube 1916 has its articulation prohibited by the proximal outer sleeve 1912 covering the articulation joints 1918. Retraction of the proximal outer sleeve 1912 simultaneously exposes sequential articulation joints 1918 and applies a tensile force on the articulation tendon 1914. As each articulation joint 1918 is exposed, it bends in the direction of the articulation tendon 1914 to its maximum articulation angle until the next sequential articulation joint 1918 is exposed.

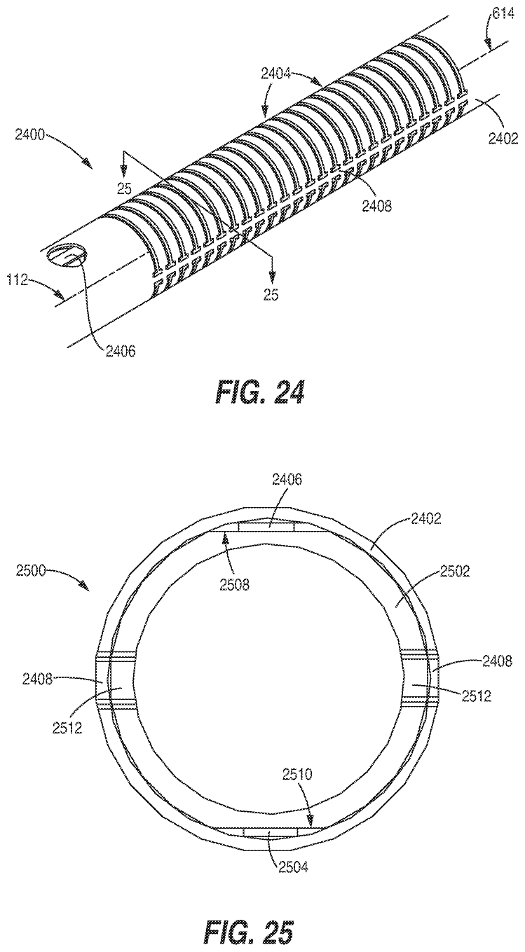

FIG. 24 depicts a perspective view of a two-tendon articulation device, in accordance with the teachings of the present disclosure. FIG. 25 depicts a cross-sectional view of the two-tendon articulation device of FIG. 24, in accordance with the teachings of the present disclosure. In particular, FIG. 24 depicts the perspective view 2400 that includes an outer flexible tube 2402 that may be similar in construction to the inner flexible tube 1916 discussed above. The aspects of the two-tendon articulation device may be used with a surgical fixation described herein, or with other similar surgical fixation devices. The outer flexible tube 2402 includes the plurality of articulation joints 2404 that permits the outer flexible tube 2402 to bend (e.g., bend up and down, articulate) and the side stiffener column 2408 that stiffens the outer flexible tube 2402 from bending (e.g., left and right). The first articulation tendon 2406 is configured to apply a tension force to bias the outer flexible tube 2402 upwards.

In the view 2500, a cross-section of the view 2400 taken at 25. It can be seen that the two-tendon articulation device includes both the outer flexible tube 2402 and an inner flexible tube 2502. The inner flexible tube 2502 is similar in construction to the outer flexible tube 2402, but includes the upper flat slot 2508 to permit the first articulation tendon 2406 to be nested between the inner and outer flexible tubes 2402, 2502 and also includes the lower flat slot 2510 to permit a second articulation tendon 2504 to be nested between the inner and outer flexible tubes 2402, 2502. The side stiffener column 2408 of the outer flexible tube 2402 is aligned with the side channel 2512 of the inner flexible tube 2502. The second articulation tendon 2504 applies a tensile force opposite of the first articulation tendon 2406. As such, balancing of the forces on the first articulation tendon 2406 and the second articulation tendon 2504 permits control of the articulation angle (an angle between the longitudinal axis 112 and the longitudinal axis 614). These tensile forces from the articulation tendons 2406, 2504 are applied to the distal end 1910 of a surgical fixation device as controlled by an articulation actuator configured to adjust the tension of both the first and second articulation tendons 2406, 2504 response to a user input to achieve the desired articulation angle. This two-tendon articulation system eliminates the need for a proximal outer sleeve to cover the articulation joints and further eliminates an articulation tendon that creates a bow external to the articulation device, like the articulation device depicted in the view 2100. Similar to the attachment points 1928, 1930 discussed in FIGS. 20 and 21 with respect to the single tendon articulation device, the first and second articulation tendons 2406, 2504 may similarly be connected either one or both of the inner flexible tube 2402 and the inner flexible tube 2502 at the distal end of the drive shaft from the articulation joints 1918. The first articulation tendon 2406 is attached on an upper side of the inner flexible tube 1916 and the second articulation tendon 2504 is attached on a lower side of the inner flexible tube 1916. The upper side is on an opposite half of the stiffener column 2202 as the lower side to allow for one of the two articulation tendons 2406, 2504 to cause the articulation of the distal end of the fastener device and the other of the two articulation tendons 2406, 2504 to allow for straightening of the distal end of the fastener device. The articulation tendons 2406, 2504 may be attached, or affixed, at the connection points via a welding process, such as a welding process. Further, proximal portions of the articulation tendons 2406, 2504 are attached to an articulation control device to alternate the application of the tensions applied to the articulation tendons 2406, 2504 to control articulation of the fastener device.

From the foregoing, it can be seen that the present disclosure sets forth a medical fastening device adapted to rapidly and reliably install fasteners to secure tissue and/or any applicable prosthetic material. The device not only greatly reduces the time required for fastening tissues, but also results in superior ease of use relative to other methods. Furthermore, through the unique combination of elements set forth in the present disclosure, the tissue fastening is more reliably retained with reduced irritation and other complications to the patient and without adversely affecting the integrity of the attachment and/or closure.

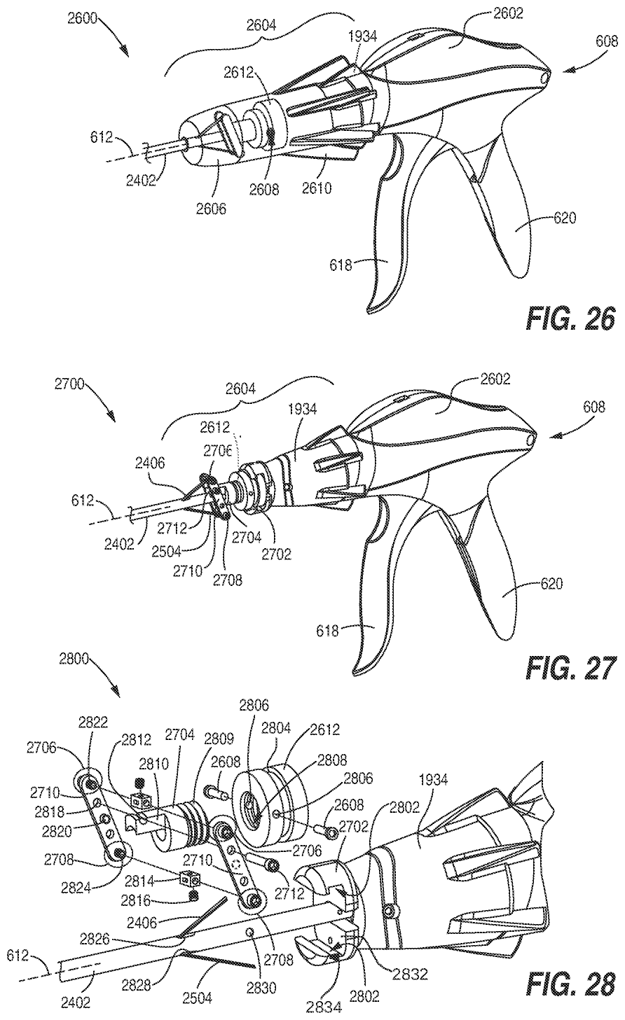

FIG. 26 depicts a perspective view of an articulation control portion of a fastener deployment device, in accordance with the teachings of the present disclosure. In particular, FIG. 26 depicts the perspective view 2600 of the fastener deployment device 2602 that may include the two-tendon articulation device that is depicted in FIGS. 24-25. The articulation control portion depicted in the FIGS. 26-31, which may also be referred to as an articulation control unit, serves as one way of controlling the articulation of an articulation device having two tendons. The fastener deployment device 2602 may be similar to that of the fastener deployment device 602, with like numbered parts having the same functions. It is further envisioned that the two-tendon articulation device, and its associated controls, could be used with a variety of other fastener deployment devices as well.

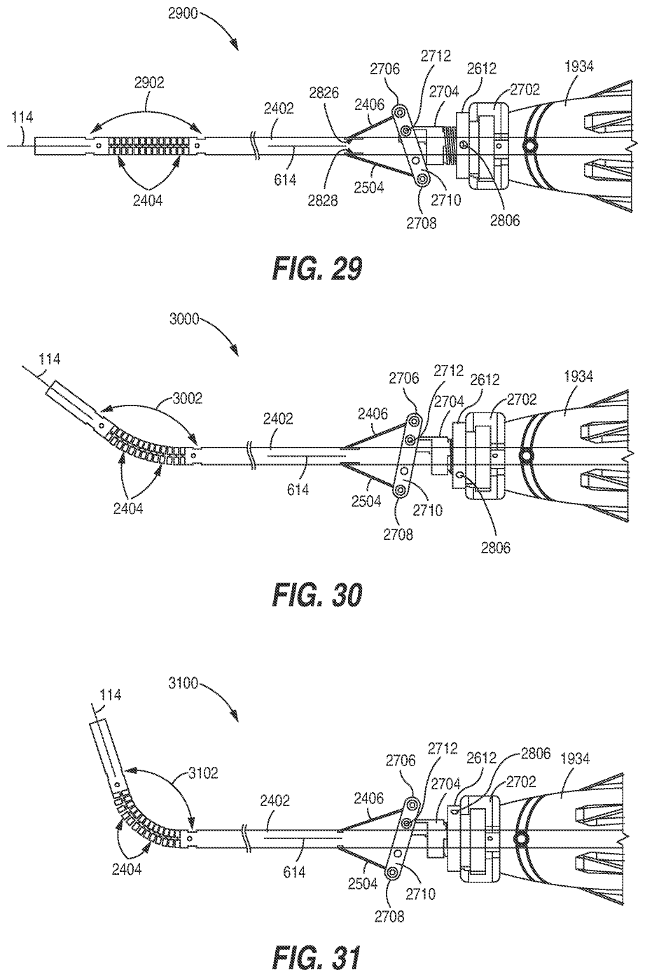

In general, aspects of articulation control portion 2604 receive an operator input at the proximal end 608 of the fastener deployment device 2602 to control an articulation angle (e.g., 2902, 3002, and 3102 of FIGS. 29-31) of the distal end of the fastener deployment device 2602. In one such embodiment, the articulation control portion 2604 increases a tension to the first articulation tendon 2406 and reduces a tension to the second articulation tendon 2504 in response to receiving a first operator input and decreases the tension to the first articulation tendon 2406 and increases a tension to the second articulation tendon 2504 in response to receiving a second operator input that is opposite of the first operator input. This alternate tensioning of the articulation tendons 2406 and 2504 causes the distal end of the fastener deployment device 2602 to articulate or to straighten, based on receiving the first or the second operator input. One such embodiment of such an articulation control portion 2604 is discussed below by way of example.

The view 2600 includes details of the articulation control portion 2604 of the fastener deployment device 2602 that includes the articulation turn knob 2606. The articulation turn knob 2606 is adjacent to the roticulation turn knob 1934 and is able to be rotated by an operator of the fastener deployment device 2602 to change a degree of articulation of the distal end of the fastener deployment device 2602, similar to that as depicted in the view 2100 of FIG. 21 and as depicted in FIGS. 29-31. The articulation turn knob 2606 may include fins 2610 extending outward to assist the operator of the fastener deployment device 2602 to obtain a proper grip on the articulation turn knob 2606. When an operator applies a torque to the articulation turn knob 2606 about the longitudinal axis 612, the articulation turn knob 2606 rotates. The articulation turn knob 2606 is fixedly connected to the articulation nut 2612 by the fixation screw 2608. Thus, rotation of the articulation turn knob 2606 is translated via the fixation screw 2608 to the articulation nut 2612 that is located internal to the articulation turn knob 2606. Further, rotation of the roticulation turn knob 1934 causes a rotation of both the articulation turn knob 2606 and the outer flexible tube 2402, which does not cause a change in the articulation angle because the articulation turn knob 2606 is not being rotated independently from the roticulation turn knob 1934.

FIG. 27 depicts a perspective view of the articulation control portion of FIG. 26 with the articulation control knob 2606 removed, in accordance with the teachings of the present disclosure. In particular, FIG. 27 depicts the perspective view 2700 of the fastener deployment device 2602 with the articulation turn knob 2606 removed to show components located internal to the articulation turn knob 2606 in an assembled state. Further, FIG. 28 depicts an exploded view of the articulation control portion of FIG. 27, in accordance with the teachings of the present disclosure. In particular, FIG. 28 depicts the exploded view 2800 of the articulation control portion 2604 from FIG. 27.

As shown in FIGS. 27 and 28, the articulation control portion 2604 includes the outer flexible tube 2402 that extends along the longitudinal axis 612. The outer flexible tube 2402 extends, from left to right in the FIGs., between the reversing brackets 2710, through the articulation lead screw 2704, through the articulation nut 2612, and into the nut bracket 2702. The nut bracket 2702 is fixed in relation to the roticulation turn knob 1934. The nut bracket 2702 includes a channel 2802 that receives a groove 2804 of the articulation nut 2612. The channel 2802 is bounded by a proximal face 2832 and a distal lip 2834 to receive the groove 2804 of the articulation nut 2612. The nut bracket 2702 permits rotation of the articulation nut 2612 about the longitudinal axis 612 but prevents the articulation nut 2612 from translating along the longitudinal axis 612. The fixation screws 2608 mate with the screw holes 2806 on the outer surface of the articulation nut 2612. While the articulation turn knob 2606 is described as being attached to the articulation nut 2612 by way of the fixation screws 2608 being inserted into the screw holes 2806, other means of attaching the articulation turn knob 2606 to the articulation nut 2612 may exist.

The articulation nut 2612 includes a threaded surface 2808 on an interior surface of the articulation nut 2612. The threaded surface 2808 is configured to engage with the threaded surface 2809 of the articulation lead screw 2704. Thus, when the articulation nut 2612 rotates about the longitudinal axis 612, it causes the articulation lead screw 2704 to translate along the longitudinal axis 612. The articulation lead screw 2704 includes an extended portion 2810 that includes a trough 2812. The shape of the extended portion 2810 permits the reversing brackets 2710 to closely sandwich the extended portion 2810 to fit within the volume of the interior of the articulation turn knob 2606. The trough 2812 receives the actuator pin 2712. The actuator pin 2712 is fixed to the actuator pin holes 2818 in the reversing brackets 2710. The actuator pin holes 2818 are offset from the trunnion 2820 that acts a pivot point for the reversing brackets 2710 when installed into the trunnion hole 2830 along the outer flexible tube 2402. Thus, when the articulation lead screw 2704 translates along the longitudinal axis 612, it causes the actuator pin 2712 to cause the reversing brackets 2710 to pivot about the trunnion 2820. Each reversing bracket 2710 may include the trunnion 2820 facing each other to interface with pair of trunnion holes 2830 disposed on opposite sides of the outer flexible tube.

As the reversing brackets 2710 pivot, a first end 2706 of the reversing bracket is biased towards a distal end of the fastener deployment device 2602 and the second end 2708 is biased away from the distal end of the fastener deployment device 2602. When the rotation of the articulation turn knob 2606 is reversed, the opposite rotation of the articulation nut 2612 causes the opposite translation of the articulation lead screw 2704, which causes the opposite biasing of the first and second ends 2706 and 2708 of the reversing brackets 2710. As shown in FIG. 27, the first end 2706 is attached to the first articulation tendon 2406 and the second end 2708 is attached to the second articulation tendon 2504. The first articulation tendon 2406 enters the outer flexible tube 2402 via the access port 2826 and the second articulation tendon 2504 enters the outer flexible tube 2402 via the access port 2828.

The articulation tendons 2406 and 2504 are attached to the respective ends of the reversing brackets 2710. As shown in FIGS. 27-28, the articulation tendons may be inserted into the block 2814 and secured to the block 2814 with the set screw 2816. The block 2814 may be secured to a first reversing bracket 2710 via the screw 2822 for securing to the first end 2706 or the screw 2824 for securing to the second end 2708. The block 2814 may further be secured to a second reversing bracket 2710 that is disposed on an opposite side of the elongated portion 2810 by a second screw, similar to the screws 2822, 2824 as depicted, but not labeled for clarity in the view 2800 of FIG. 28. A similar attachment may be used for both the first and second ends 2706 and 2708. In some embodiments, the first and second ends 2706 and 2708 are located an equal distance from the trunnion 2820 that acts as the pivot point for the reversing bracket 2710. In such embodiments, the articulation tendons 2406 and 2504 are displaced an equal but opposite magnitude as the reversing brackets 2710 pivot. In other embodiments, the first and second ends 2706 and 2708 are located at different distances from the trunnion 2820. Such a difference between the ends 2706, 2708 and the trunnion 2820 provides for one end to be displaced a greater magnitude than the other end. This may ensure that both the articulation tendons 2406 and 2504 maintain equal tensions through articulation and straightening of the distal end as the articulation tendon being on an inner-radius of the articulation angle extends a shorter effective length than the articulation tendon being on an outer-radius of the articulation angle.

### INDUSTRIAL APPLICABILITY

In general, the embodiments disclosed herein can find applicability in various suturing and surgical applications, including, but not limited to, fastening tissues and meshes in the context of surgery. The surgical fasteners 102 disclosed herein may be flexible and/or resorbable fasteners that have at least one helical arm extending from its base. The surgical fasteners disclosed herein are inserted into the mesh and/or tissue by way of a needle that advances along the at least one helical arm of the surgical fastener. The needle engages with a needle capture zone of the surgical fastener and pulls the surgical fastener to install it within the tissue and/or mesh. The needle subsequently retracts along the helical path it advanced.

In the different embodiments, the various surgical fasteners 102 disclosed in conjunction with FIGS. 1-5 may be installed to fasten a mesh to a tissue, a tissue to a tissue, or the like by any one of the fastener deployment devices described herein. Further, the fastener deployment devices disclosed herein may be equipped with the impulse energy drive mechanism 616. In yet other embodiments, the fastener deployment devices may be configured to articulate. One such embodiment with articulation is disclosed in conjunction with the embodiments depicted in FIGS. 19-23 having a single articulation tendon. In yet other embodiments, the fastener deployment device includes a two-tendon articulation device, such as the articulation device depicted at least in part in FIGS. 24-28.

FIG. 29 depicts the fastener deployment device of FIG. 26 in an unarticulated position, FIG. 30 depicts the fastener deployment device of FIG. 26 in a partially articulated position, and FIG. 31 depicts the fastener deployment device of FIG. 26 in a fully articulated position, in accordance with the teachings of the present disclosure.

In particular, FIG. 29 depicts the view 2900, FIG. 30 depicts the view 3000 and FIG. 31 depicts the view 3100, each showing a two-tendon articulation device having its articulation turn knob 2606 removed for clarity purposes. The two-tendon articulation device depicted herein may be utilized on a multitude of different surgical devices, including the fastener deployment devices described herein, laparoscopic devices, and the like.

In the view 2900 of FIG. 29, the distal end of the fastener deployment device is not articulated, meaning that the longitudinal axis 114 coincides with the longitudinal axis 614, and the articulation angle 2902 measures 180 degrees. Here, the first end 2706 of the reversing bracket 2710 is disposed towards the distal end of the fastener deployment device and the second end 2708 of the reversing bracket 2710 is disposed towards the proximal end of the fastener deployment device. The first articulation tendon 2406 extends out from the outer flexible tube 2402 via the access port 2826 and is fixed to the first end 2706. The second articulation tendon 2504 extends out from the outer flexible tube 2402 via the access port 2828 and is fixed to the second end 2708. One or both of the articulation tendons 2406, 2504 may be under tension when at the initial state depicted in the view 2900. A drive mechanism may actuate installation of a surgical fastener from the distal end in the non-articulated position.

The view 3000 depicts the fastener deployment device in a partially articulated position. Here, a user input (e.g., a user rotating the not-depicted articulation turn knob 2606) causes the articulation nut 2612 to rotate within the nut bracket 2702, as depicted at least in part by the change in location of the screw hole 2806 as compared to the location of the screw hole 2806 as depicted in the view 2900. In the view 2900, a first screw hole 2806 is depicted and in the view 3000, a second screw hole—located opposite from the first screw hole—is depicted. Thus, the first screw hole 2806 has rotated out of the view 3000 and the second screw hole 2806 is visible. The rotation of the articulation nut 2612 causes translation of the articulation lead screw 2704 along the longitudinal axis 614. Here, it has translated in the proximal direction and into the articulation nut 2612. The trough 2812 pulls the actuator pin 2712 in the proximal direction to bias the first end 2706 in the proximal direction. This pivots the reversing bracket 2710 about the trunnion 2820 to cause the second end 2708 to bias towards the distal end. This motion of the reversing bracket 2710 increases the tension of the first articulation tendon 2406 and releases the tension on the second articulation tendon 2504. The increased tension on the first articulation tendon 2406 causes the articulation joints 2404 to bend and imparts the articulation angle 3002 between the longitudinal axis 114 and the longitudinal axis 614. As depicted in the view 3000, the articulation angle 3002 is approximately 150 degrees.

In the view 3100, the fastener deployment device is depicted under full articulation. Here, the articulation turn knob 2606 has been further rotated as compared to the view 3000. This caused further rotation of the articulation nut 2612, further translation of the articulation lead screw 2704, and further pivoting of the reversing brackets 2710 to apply more tension on the first articulation tendon 2406 and to further let out the second articulation tendon 2504. The fastener deployment device may reach maximum articulation based on a maximum bend of the articulation joints 2404, by way of the articulation nut 2612 reaching a hard stop, or by the actuator pin 2712 reaching a hard stop within the trough 2812. As depicted in the view 3100, the articulation angle 3102 is approximately 110 degrees. Surgical fasteners (e.g., 102) may be installed into a tissue and/or mesh at the articulation angles 3000, 3002 and 3102, or any other angle in between.

To straighten the fastener deployment device, the user may apply an opposite user input to the articulation turn knob 2606. Here, the user would apply an opposite turn force to the articulation turn knob to reverse the process depicted sequentially through the views 2900, 3000, and 3100. With the opposite turn force applied, the articulation nut 2612 rotates in an opposite direction to cause the articulation lead screw 2704 to translate towards the distal end of the fastener deployment device. This causes the trough 2812 to push the actuator pin 2712 to cause the reversing bracket 2710 to pivot about its trunnion 2820. The first end 2706 is then biased towards the proximal end of the fastener deployment device and the second end 2708 is biased towards the distal end of the fastener deployment device. This increases the tension on the second articulation tendon 2504 and reduces the tension on the first articulation tendon 2406 to cause the distal end of the fastener deployment device to straighten out. Thus in this example, the opposite user input to the articulation turn knob 2606 transitions the fastener deployment device from the view 3100 of maximum articulation angle, to the view 300 of an intermediate articulation angle, to the view 2900 depicting no articulation angle.

It is noted that in embodiments with a single articulation tendon, such as depicted in the view 2100 of FIG. 21, the articulation tendon 1914 extends between the points 1928 and 1930, outside of the proximal outer sleeve 1912 and the distal outer sleeve 1922. Thus, the tendon 1914 extends outside of the articulation device when inserted into a patient when performing the fastening laparoscopically. However, in the two-tendon articulation device embodiments depicted in the views 2900-3100 of FIGS. 29-31, the two tendons are both internal to the outer flexible tube 2402 at locations that would be inside of the patient when performing the fastening laparoscopically.

In embodiments having the articulation lead screw 2704 and the articulation nut 2612, it is expected that a straightening force or a bending force applied to the distal end of the fastener deployment device would be insufficient to cause rotation of the articulation nut. Thus, an application of a usual force to the distal end of the fastener deployment device would not would result in straightening or articulation of the distal end based at least in part on the lack of a mechanical advantage attainable at the distal end of the fastener deployment device as compared to the pitch on the articulation lead screw 2704 and the articulation nut 2612.

What is claimed is:

1. A surgical fastener comprising:
a base at a proximal end of the surgical fastener;
a first arm and a second arm, the first arm and the second arm extending helically from the base about a longitudinal axis towards a distal end of the surgical fastener;
a first needle capture zone disposed on a distal portion of the first arm; and
a second needle capture zone disposed on a distal portion of the second arm, wherein the first needle capture zone and the second needle capture zone are configured to engage with a respective first needle and a second needle when the first and the second needle are extending from a fastener deployment device and disengage with the respective first and second needles when the first needle and the second needle are retracting into the fastener deployment device.

2. The surgical fastener of claim 1, further comprising a retention device disposed along each of the first arm and the second arm between the proximal end and the distal end, wherein an outer circumferential surface of the first arm and the second arm is smooth, and the retention device is disposed on a side surface of each of the first arm and the second arm.

3. The surgical fastener of claim 1, wherein the surgical fastener is a resorbable fastener.

4. The surgical fastener of claim 1, wherein the surgical fastener is a flexible fastener.

5. The surgical fastener of claim 1, wherein the base comprises a circular ring.

6. The surgical fastener of claim 1, wherein the base comprises a semi-circle.

7. The surgical fastener of claim 1, wherein the base includes a de-rotation locking feature extending from the base to interact with a fastening interface to resist a de-rotation of the surgical fastener when installed in either one of a mesh or a tissue.

8. The surgical fastener of claim 1, wherein the first arm extends from a first point of the base and the second arm extends from a second point of the base, the first point being disposed across from the second point.

9. The surgical fastener of claim 1, wherein the first arm and the second arm extend helically from the base at a pitch, and the first and second needles are configured to extend from the fastener deployment device at the pitch.

10. The surgical fastener of claim 1, wherein the first and second arms form a circular double helix shape.

11. The surgical fastener of claim 1, wherein a length of the surgical fastener corresponds to an axial range of extension of the first and second needles extending from the fastener deployment device.

12. The surgical fastener of claim 1, wherein a first tip of the first needle and a second tip of the second needle are configured to extend through the respective first and second needle capture zones to penetrate a tissue.

13. The surgical fastener of claim 1, wherein:
the first needle capture zone and the second needle capture zone are configured to enclose a respective tip of the first and second needles;
and each of the first and second needle capture zones further comprise a piercing tip.

14. The surgical fastener of claim 1, wherein each of the first and second needle capture zones are configured to pull the surgical fastener through a tissue.

15. The surgical fastener of claim 1, wherein the first arm and the second arm complete one full revolution about the longitudinal axis.

16. A method of installing a surgical fastener in a tissue, the method comprising:
loading the surgical fastener at a needle interface of a fastener deployment device;
extending a first needle and a second needle along a first helical arm and a second helical arm of the surgical fastener;
engaging the first needle with a first needle capture zone disposed on a distal portion of the first helical arm and engaging the second needle with a second needle capture zone disposed on a distal portion of the second helical arm;
pulling, by the first and second needle capture zones, the surgical fastener through the tissue; and
retracting the first and second needles to disengage the first and second needles from the respective first and second needle capture zones.

17. The method of claim 16, wherein extending the first needle and the second needle along the first helical arm and the second helical arm comprises rotationally and axially extending the first and second needles.

18. The method of claim 16, further comprising penetrating, by one of a piercing tip of the surgical fastener and tips of the first and second needles, one of the tissue and a mesh.

19. A fastener deployment device comprising:
a first spiral needle and a second spiral needle, the first and second spiral needles disposed at a distal end of the fastener deployment device;
a drive shaft extending along a longitudinal axis, the drive shaft being disposed within an outer sleeve and coupled to the first and second spiral needles;
a drive mechanism at a proximal end of the fastener deployment device, the drive mechanism being operatively coupled to the drive shaft and configured to extend both the first and second spiral needles axially along and radially around the longitudinal axis simultaneously, wherein the fastener deployment device is configured to install a surgical fastener, the surgical fastener comprising:
a base at a proximal end of the surgical fastener;
a first arm and a second arm, the first arm and the second arm extending helically from the base about the longitudinal axis towards a distal end of the surgical fastener;
a first needle capture zone disposed on a distal portion of the first arm; and
a second needle capture zone disposed on a distal portion of the second arm, wherein the first needle capture zone and the second needle capture zone are configured to engage with the first spiral needle and the second spiral needle when the first and second spiral needles are extending from the fastener deployment device and disengage from the respective first and second spiral needles when the first and second spiral needles are retracting into the fastener deployment device.

20. The fastener deployment device of claim 19, wherein the fastener deployment device further comprises:
the drive shaft comprising an inner flexible tube and an outer flexible tube having a plurality of articulation joints configured to permit articulation of the distal end of the fastener deployment device;
a first articulation tendon disposed between the inner flexible tube and the outer flexible tube, the first articulation tendon being affixed to one or both of the inner flexible tube and the outer flexible tube at a first point distal from the plurality of articulation joints;
a second articulation tendon disposed between the inner flexible tube and the outer flexible tube, the second articulation tendon being affixed to one or both of the inner flexible tube and the outer flexible tube at a second point, the second point being distal from the plurality of articulation joints and disposed on an opposite side of the longitudinal axis from the first point; and
an articulation control unit configured to:
apply a first tension to one of the first and the second articulation tendons to articulate the distal end of the drive shaft about the plurality of articulation joints; and
apply a second tension to the other of the first and the second articulation tendons to straighten the distal end of the drive shaft.
